(19) United States
(12) Patent Application Publication
(10) Pub. No.: US 2004/0053292 A1
(43) Pub. Date: Mar. 18, 2004

(54) Proteins and dna sequences underlying these proteins used for treating inflammations

(76) Inventors: Jurg Tschopp, ch. des Fontannins (CH); Fabio Martinon, Valentin (CH)

Correspondence Address: CONNOLLY BOVE LODGE & HUTZ, LLP, P O BOX 2207, WILMINGTON, DE, 19899, US

(21) Appl. No.: 10/416,935

(22) PCT Filed: Oct. 30, 2001
(86) PCT No.: PCT/EP01/12545

(30) Foreign Application Priority Data
Nov. 15, 2000 (DE) 100 56 687.1
Nov. 30, 2000 (DE) 100 59 595.2

(51) Int. Cl. C12Q 1/68; C07H 21/04; C07K 14/47

(52) U.S. Cl. 435/6; 435/69.1; 435/320.1; 435/325; 530/350; 536/23.5

(57) ABSTRACT

The invention relates to DNA sequences, which code for at least one PYD domain, to expression vectors, which contain DNA sequences of this type, to host cells, which are transformed using expression vectors of this type, to purified gene products of said DNA sequences, to antibodies directed against said gene products, and to methods for isolating and/or expressing said gene products. The invention also relates to the use of said DNA sequences or of their gene products for treating inflammations.

The present invention relates to DNA sequences which code for at least one PYD domain, to expression vectors which comprise such DNA sequences, to host cells which are transformed with such expression vectors, to purified gene products of the aforementioned DNA sequences, to antibodies against the aforementioned gene products, to methods for the isolation and/or for the expression of the aforementioned gene products and to the use of the DNA sequences or of the gene products for the treatment of inflammatory events.

Proteins have a modular structure, with the individual sections of proteins being structurally and, where appropriate, functionally independent. These sections are called domains. Proteins with a modular structure occur for example in proteins of the apoptotic signal transduction chain (Aravind et al. (1999) TIBS, 24, 47-53; Hofmann (1999) Cell. Mol. Life. Sci., 55, 1113-28). For the class of apoptotic signal transduction proteins, mention may be made of three families of domains, namely the family of death domains (DD), the family of death effector domains (DED) and the family of the caspase recruitment domain (CARD), all of which are remotely related to one another and all of which also belong to a superfamily which typically forms in its 3-dimensional structure a bundle of six helices (“six helix bundle”). Proteins having a death domain, death effector domain and/or a CARD domain include, for example, the proteins FLIP, CARDIAK-RIP2, ARC, Bcll0, DEDD, as shown in the publications by Irmler et al., 1997, Nature, 388, 190-195; Koseki et al. 1998, Proct. Natl. Acad. Sci. USA, 95, 5156-60; McCarthy et al. 1998, J. Biol. Chem. 273, 16968-16975; Stegh, et al., 1998, EMBO J., 17, 5974-86; Thome et al., 1999, J. Biol. Chem., 274, 9962-8. Thus, whereas numerous proteins having domains of the structural superfamily (bundle of six helices) are involved in intracellular signal transduction processes, in particular because of their ability to associate with upstream proteins or proteins downstream in signal transduction, the proteins involved in the inflammatory response are, just like the form of the signal transmission in inflammatory responses, substantially unknown.

It is an object of the present invention firstly to identify such proteins (with their amino acid sequences) and, where appropriate, the underlying DNA sequences which are involved in the inflammatory response, or to assign a function in the inflammatory response cascade to proteins known from another context, and secondly to provide substances for the treatment of inflammatory responses through determination of the signal transduction mechanisms.

To achieve this object, the inventors have firstly found that PYD domains play a crucial role in the intracellular transmission of an inflammatory signal. It has been found according to the invention that the PYD domain likewise has the structure of the bundle of 6 helices and that it is comparable in its interaction potential with the DED, DD or CARD domains which are structurally related in this regard. Thus, the invention provides a fourth family of “six helix bundle” domains (referred to herein as “PYD” domain) which occurs as a constituent of native proteins.

The present invention therefore relates to DNA sequences which code for a protein having at least one PYD domain, including all functionally homologous derivatives, fragments or alleles. In particular, all DNA sequences which hybridize with the DNA sequences of the invention, including the sequences complementary in the double strand in each case, are also included (claim 1).

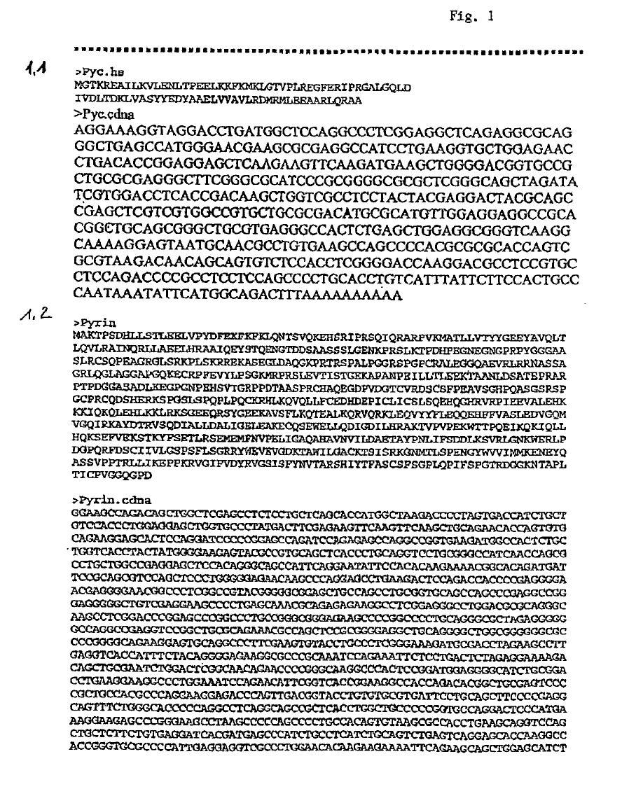

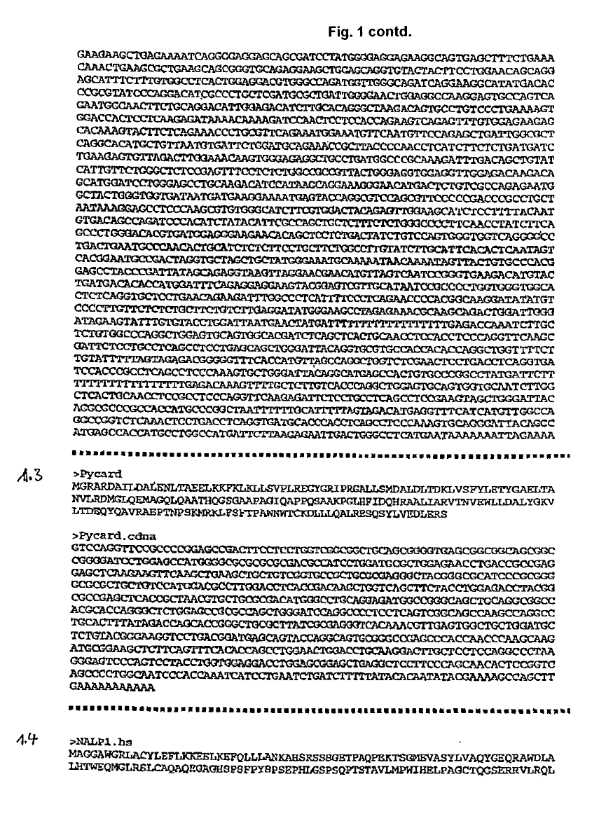

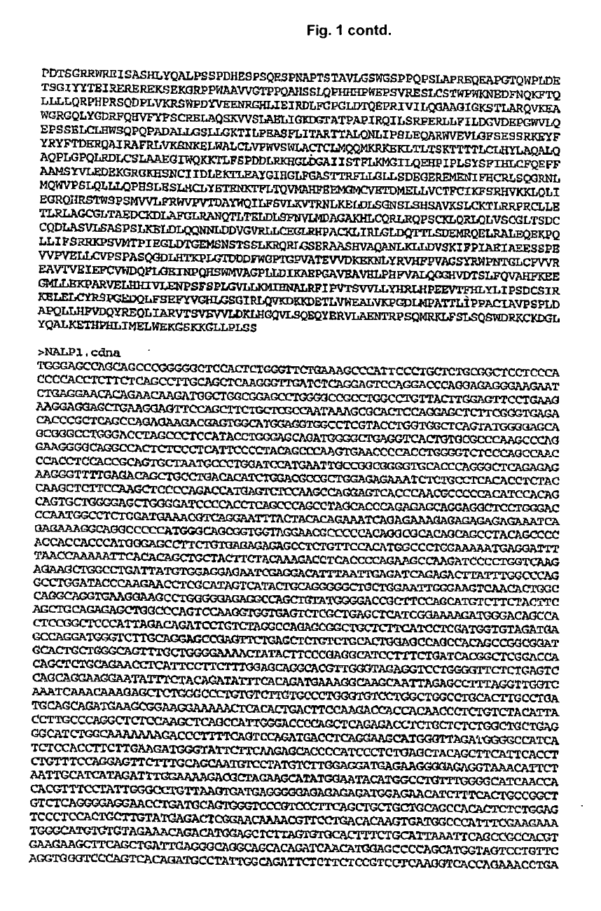

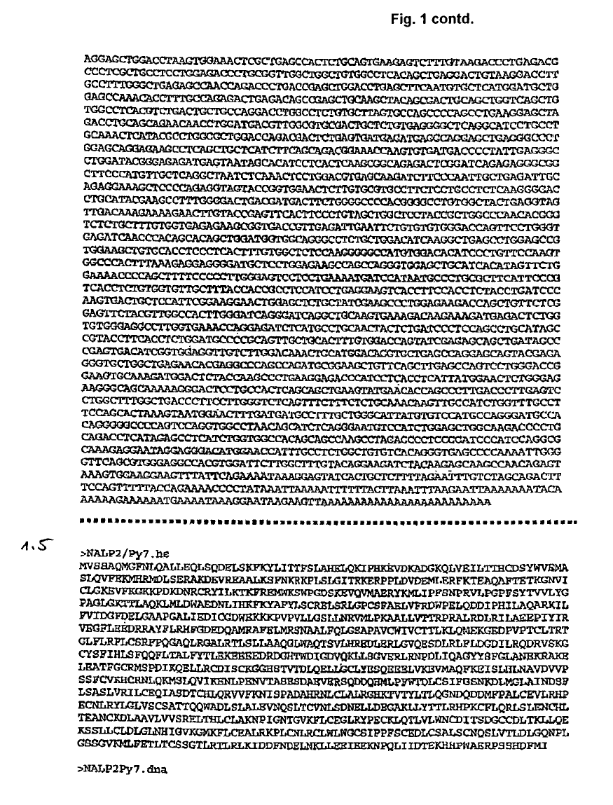

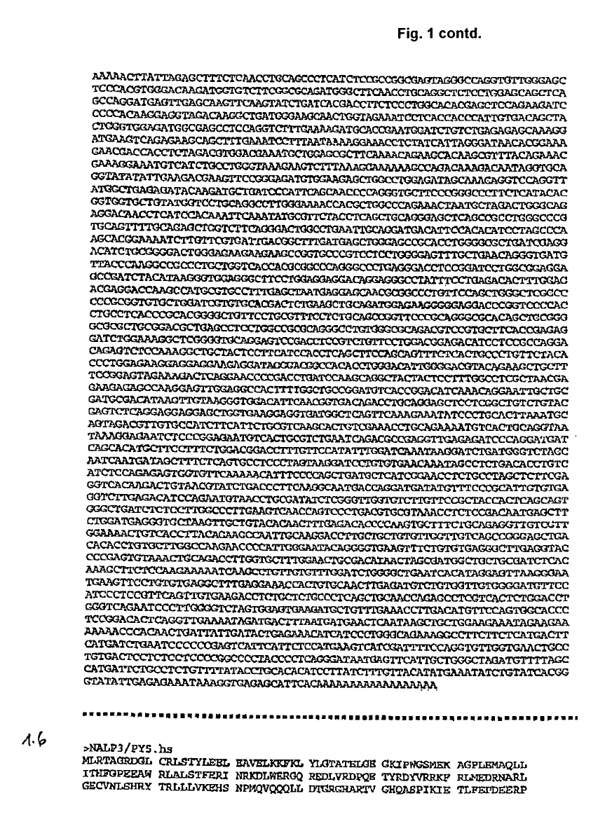

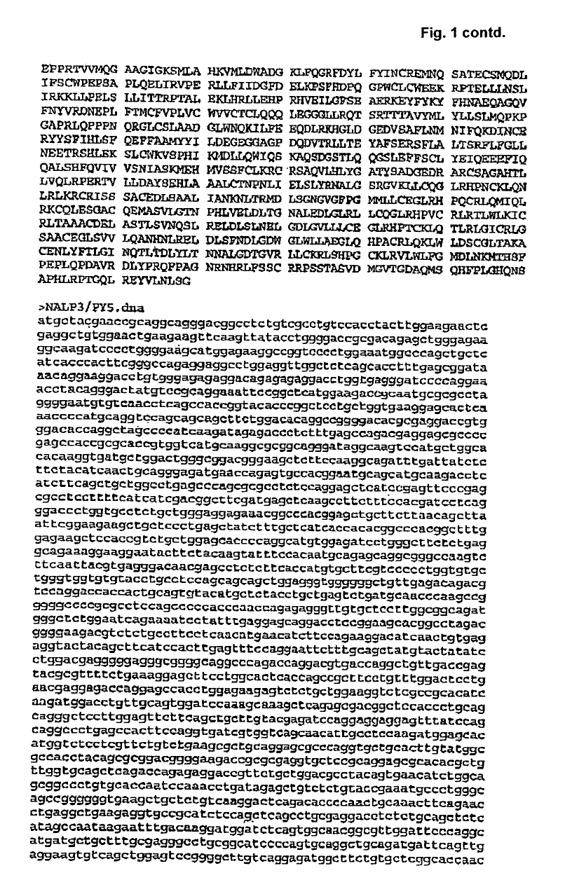

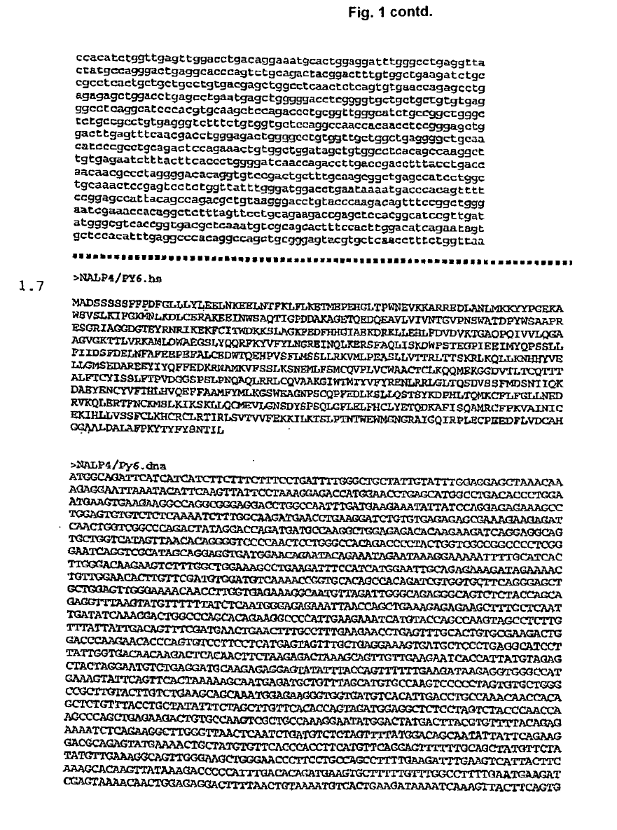

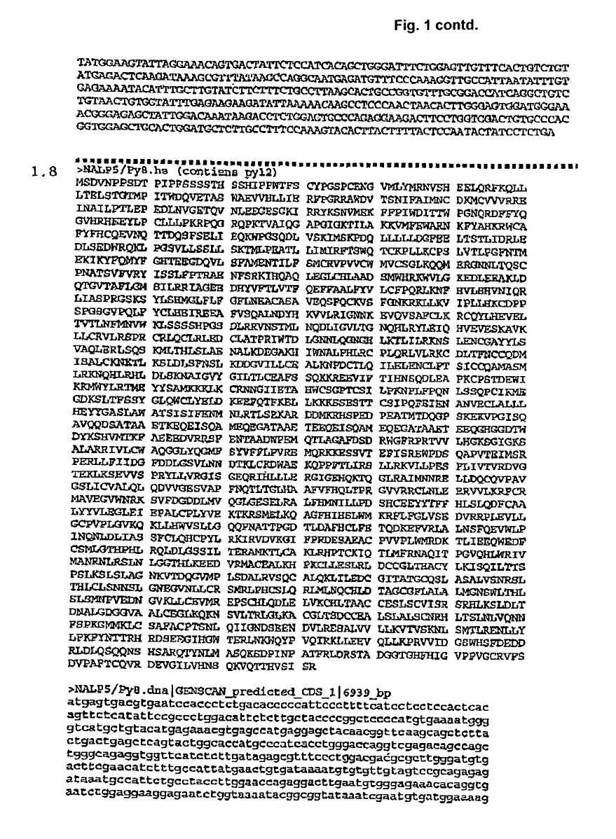

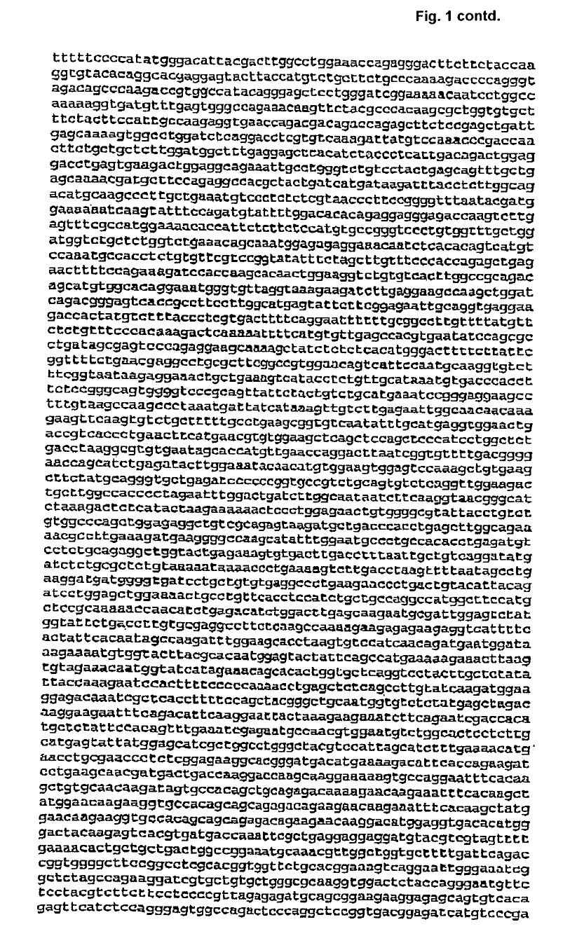

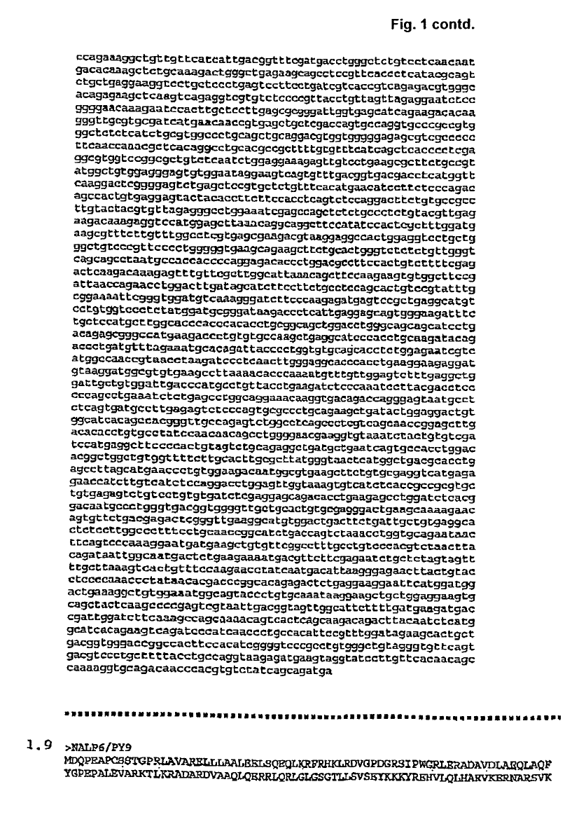

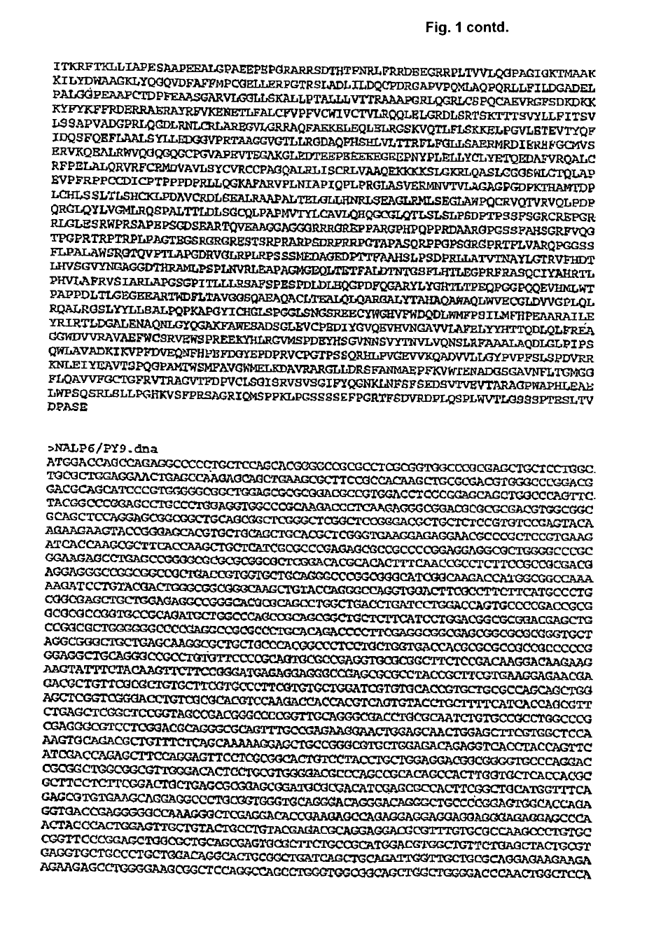

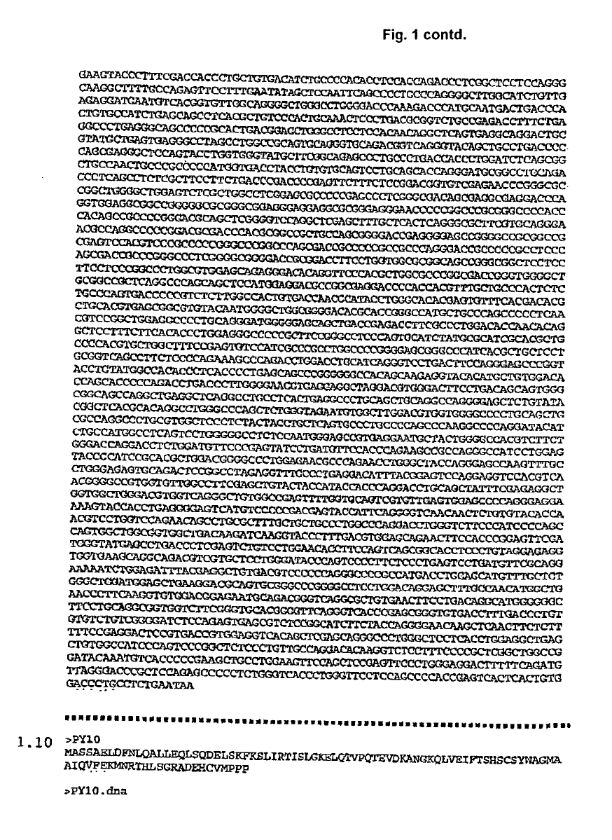

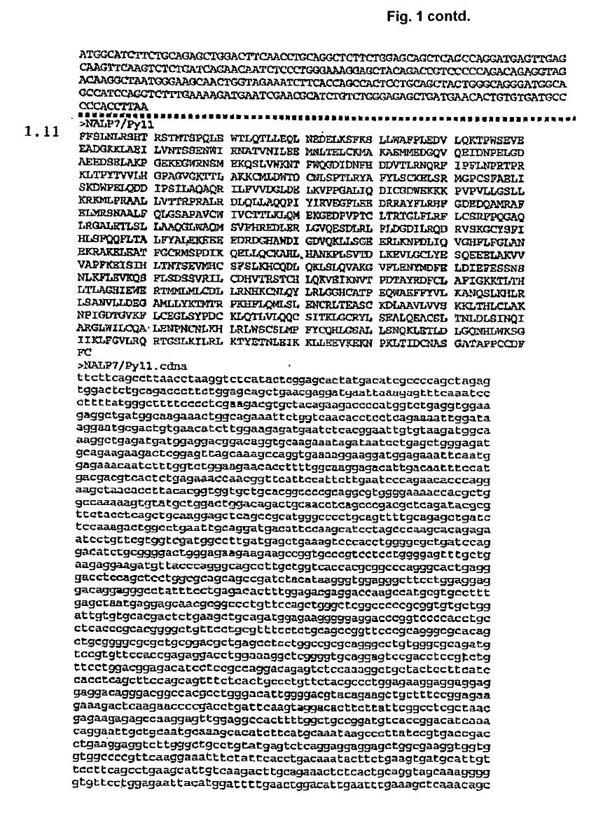

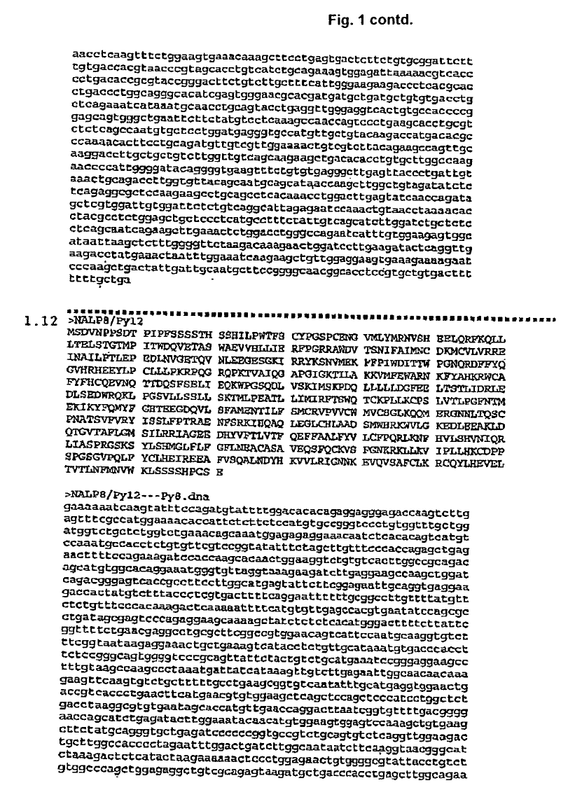

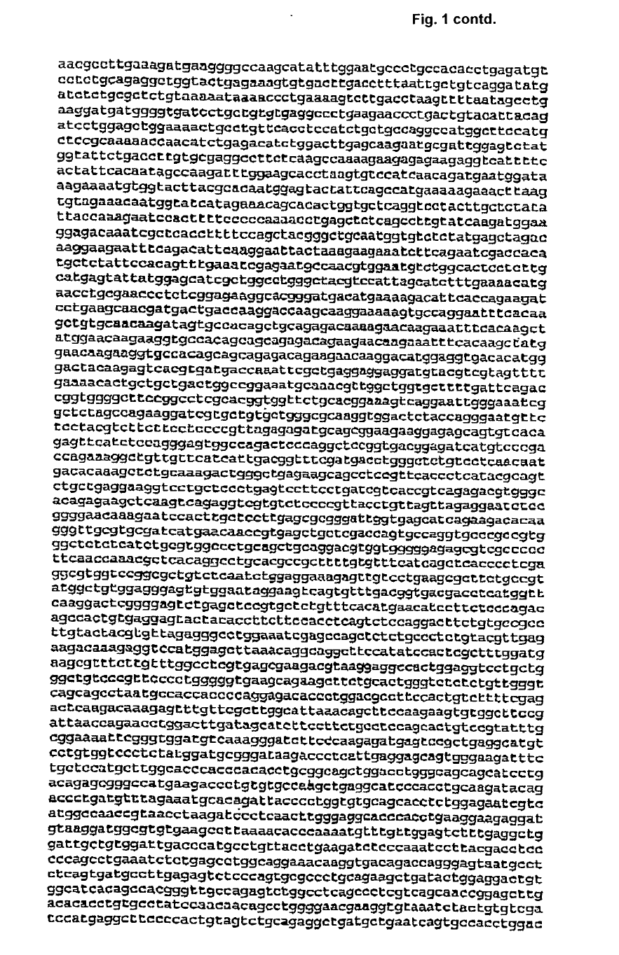

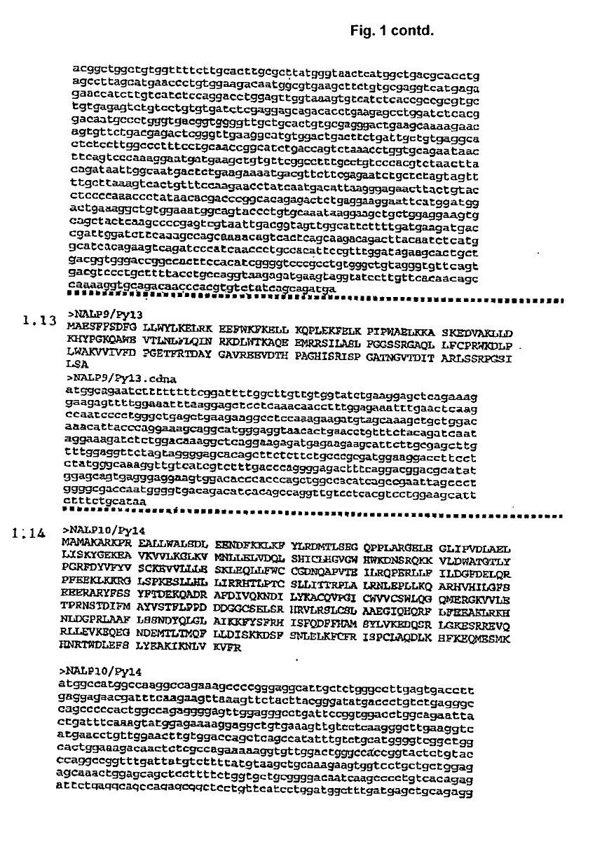

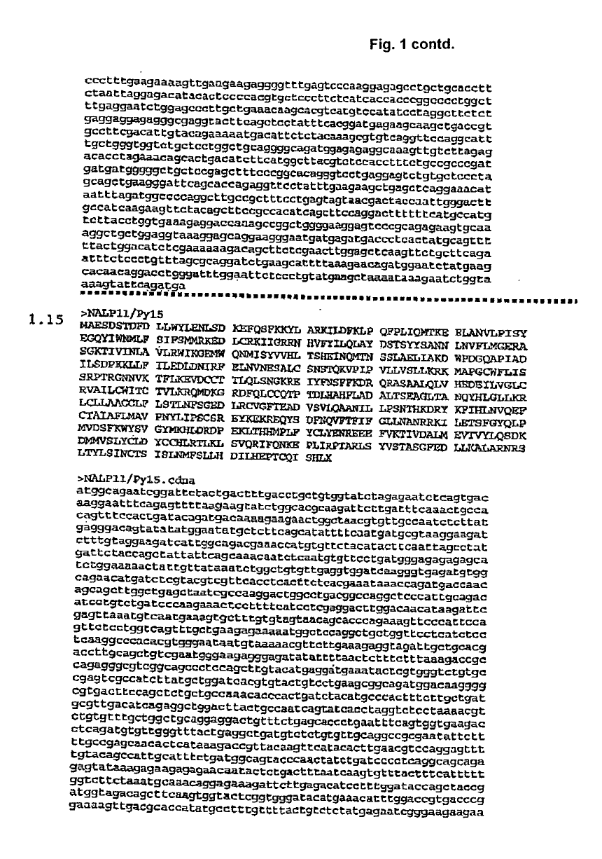

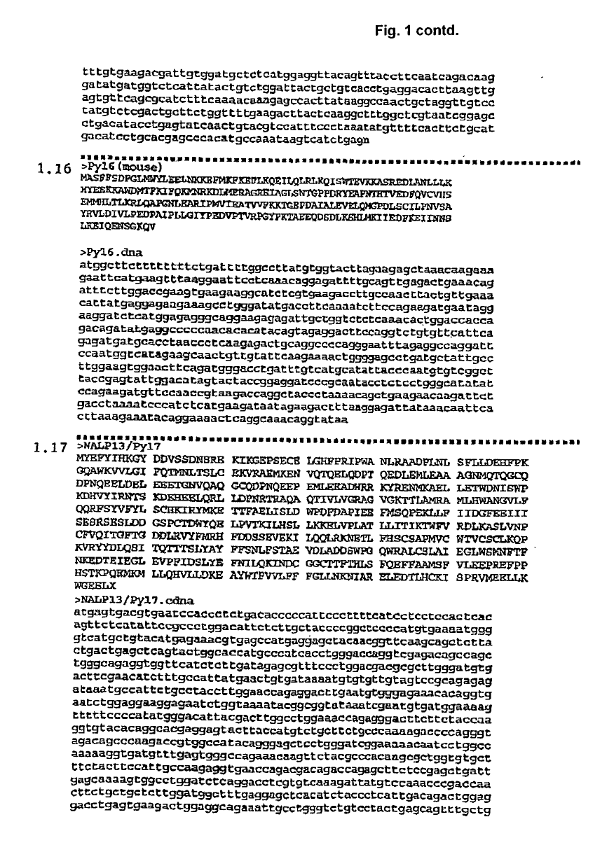

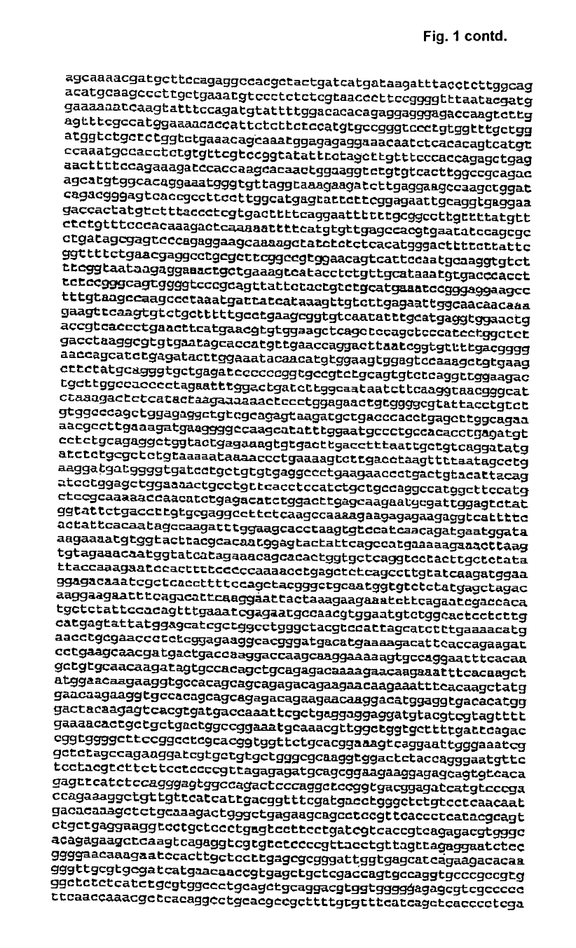

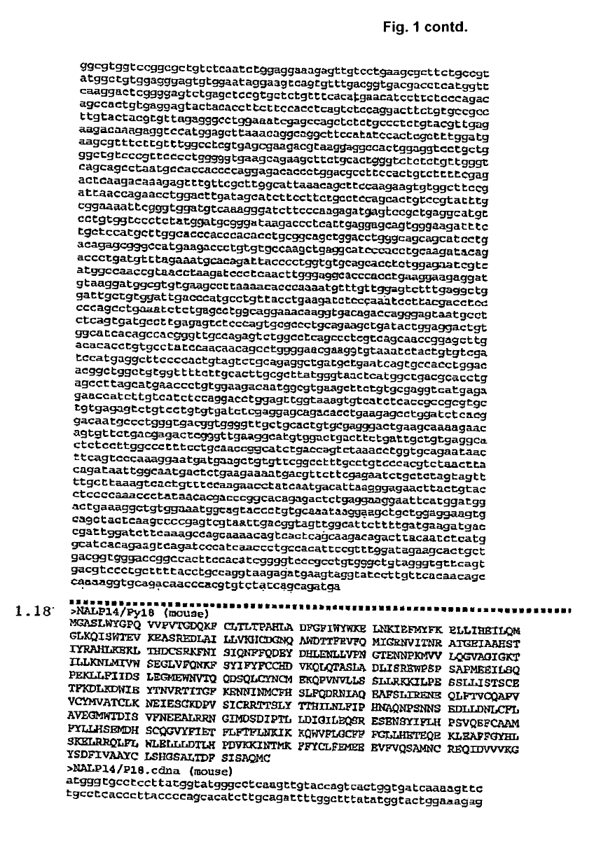

In relation to the hydridization conditions, it is disclosed specifically that homologous or sequence-related DNA sequences are isolated from all mammalian species, including humans, by conventional methods through homology screening by hybridization with a sample of the nucleic acid sequences of the invention or parts thereof. Functional equivalents are also to be understood to mean sequences comprising homologs of the native PYD domains, for example of the sequences depicted in FIG. 1, for example their homologs from other mammals, truncated sequences, single-stranded DNA or RNA of the coding and noncoding DNA sequence.

It is advantageous to use for the hybrization short oligonucleotides of the conserved regions, which can be established in a manner known to the skilled worker. In every case, the use and function of at least 15, preferably at least 20 AA-long nucleotide sections (also disclosed as such) of the nucleotide sequences contained in figure as primers for PCR reactions or as oligonucleotides on DNA chips is disclosed. However, it is also possible to use longer fragments of the nucleic acids of the invention or the complete sequences for the hybridization. These standard conditions vary depending on the nucleic acid sequence used (oligonucleotide, longer fragment or complete sequence), and depending which type of nucleic acid (DNA or RNA) are used for the hybridization. Thus, for example, the melting temperatures for DNA:DNA hybrids are about 10° C. lower than those of DNA:RNA hybrids of the same length. Standard conditions mean, for example, depending on the nucleic acid, temperatures between 42 and 58° C. in aqueous buffer solution with a concentration between 0.1 to 5×SSC (1×SSC=0.15 M NaCl, 15 mM sodium citrate, pH 7.2) or additionally in the presence of 50% formamide, such as, for example, 42° C. in 5×SSC, 50% formamide. It is advantageous for the hybridization conditions for DNA:DNA hybrids to be 0.1×SSC and temperatures between about 20° C. to 45° C., preferably between about 30° C. to 45° C. The hybridization conditions for DNA:RNA hybrids are advantageously 0.1×SSC and temperatures between about 30° C. to 55° C., preferably between about 45° C. to 55° C. These stated temperatures for the hybridization are melting temperatures calculated by way of example for a nucleic acid with a length of about 100 nucleotides and a G+C content of 50% in the absence of formamide. The experimental conditions for DNA hybridization are described in relevant textbooks of genetics such as, for example, in Sambrook et al. (“Molecular Cloning”, Cold Spring Harbor Laboratory, 1989), and can be calculated by formulae known to the skilled worker, for example depending on the length of the nucleic acids, the nature of the hybrids or the G+C content. Further information on the hybridization can be found by the skilled worker in the following textbooks: Ausubel et al. (eds), 1985, Current Protocols in Molecular Biology, John Wiley & Sons, New York; Hames and Higgins (eds), 1985, Nucleic Acids Hybridization: A Practical Approach, IRL Press at Oxford University Press, Oxford; Brown (ed), 1991, Essential Molecular Biology: A Practical Approach, IRL Press at Oxford University Press, Oxford.

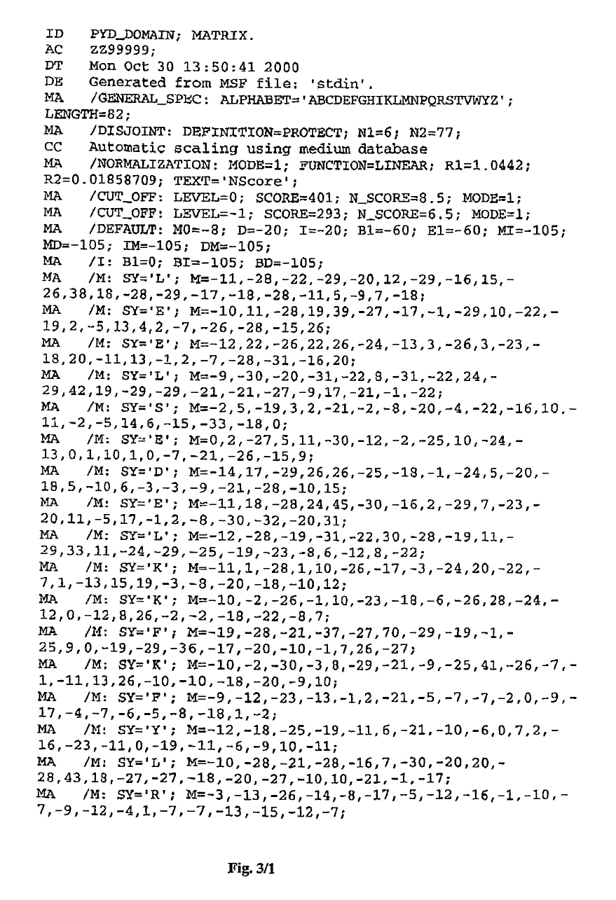

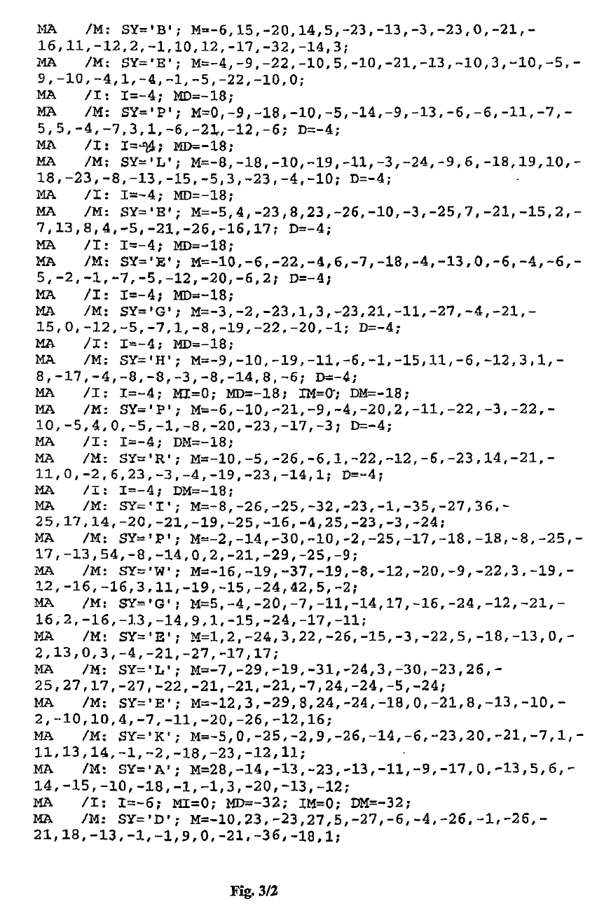

The DNA sequences of the invention disclosed in a preferred embodiment are those for which a significance level of p<10⁻² results when the PYD domain of a target DNA sequence (that is to say a DNA sequence potentially of the invention) is compared with a search profile as shown in FIG. 3 (claim 2). In relation to the experimental procedure in this regard, reference is made to the publication by Bucher et al. (1996, Computer Chem., 20, 3-24) and to the corresponding explanations in the published German patent application DE 197 13 393.2-41, both of which are incorporated to this extent in the disclosure of the present application.

The DNA sequences disclosed in a further preferred embodiment are those whose gene product comprises one of the amino acid sequences (for a PYD domain) as represented in FIG. 6, including all functionally homologous derivatives, alleles or fragments. It is advantageous for such derivatives or alleles to have a sequence homology of at least 80%, preferably of at least 90% and even more preferably of at least 95% and most preferably of at least 98% with the aforementioned sequence. DNA sequences hybridizing with these DNA sequences of the invention (including the sequences of the complementary DNA strand) are also disclosed (claim 3).

Preference is further given to DNA sequences which comprise one of the (c)DNA sequences indicated in FIG. 1 (claim 4). This is because it was found during the studies of the invention that DNA sequences of the invention code for numerous proteins (amino acid sequences) having a PYD domain which are, in particular, also involved in the inflammatory event which is pathophysiological where appropriate. These DNA sequences (including the corresponding amino acid sequences) are depicted in FIG. 1. These include, as depicted in FIG. 1 in each case with code number, the proteins Pyrin (see corresponding protein and cDNA sequences in FIG. 1, code number 1.2), Pycard (protein and cDNA sequence, code number 1.3), Pyc (protein sequence and cDNA sequence, code number 1.1), NALP1 (protein sequence and cDNA sequence, code number 1.4), NALP2 ((old name Py7) with protein sequence and one DNA sequence, code number 1.5), NALP3 ((old name PY5) with protein sequence and DNA sequence, code number 1.6), NALP4 ((old name Py6) with protein sequence and DNA sequence, code number 1.7), NALP5 ((old name Py8) with protein sequence and DNA sequence, code number 1.8), NALP6 ((old name Py9) with protein sequence and DNA sequence, code number 1.9), PY10 (with protein sequence and DNA sequence, code number 1.10), NALP7 ((old name Py11) with protein sequence and cDNA sequence, code number 1.11), NALP8 ((old name Py12) with protein sequence and DNA sequence, code number 1.12), NALP9 ((old name Py13) with protein sequence and cDNA sequence, code number 1.13), NALP10 ((old name Py14) with protein sequence and DNA sequence, code number 1.14), NALP11 ((old name Py15) with protein sequence and cDNA sequence, code number 1.15), Py16 ((murine), with protein sequence and DNA sequence, code number 1.16), NALP13 ((old name Py17), with protein sequence and cDNA sequence, code number 1.17), NALP14 ((old name Py18), murine, with protein sequence and cDNA sequence, code number 1.18), NALP15 ((old name Py19) with protein sequence and partial DNA sequence, code number 1.19), NALP12 ((old name Py20), murine, with protein sequence and cDNA sequence, code number 1.20). The aforementioned sequences are all laid out in FIG. 1 and can be found therein under the aforementioned designations and the code numbers. The DNA and protein sequences which belong together, i.e. are comprised by one code number, are in each case separated by dotted lines in bold print from the next unit. FIG. 1 comprises 22 consecutively numbered pages. Thus a further preferred aspect of the present invention comprises DNA sequences which code for one of the gene products depicted in FIG. 1 (i.e. for one of the amino acid sequences present in FIG. 1), or DNA sequences which code at least in one section of the complete sequence for one of the amino acid sequences indicated in FIG. 1 (claim 5).

The present invention further relates to expression vectors which comprise a DNA sequence of the invention, for example as disclosed previously or as claimed in claims 1 to 5 (claim 6). Such expression vectors of the invention (for example plasmids) comprise, besides at least one DNA sequence of the invention, typically also promoter regions and terminator regions, where appropriate also marker genes (for example antibiotic resistance genes) and/or signal sequences for transport of the translated protein.

The present invention further relates to host cells which have been transformed with an expression vector of the invention (claim 7). Suitable host cells for the cloning or expression of the DNA sequences of the invention are prokaryotic yeasts or higher eukaryotic cells. In the case of prokaryotes, Gram-negative or Gram-positive organisms are expressly included. Mention should be made here of E. coli or bacilli. Host cells disclosed as preferred for the cloning of the DNA sequences of the invention are the strains E. coli 294, E. coli B and E. coli X1776, and E. coli W3110. Bacillus subtilis, Salmonella typhimurium or the like stand in the case of the bacilli. As already mentioned above, the expression vectors typically comprise a signal sequence for transport of the protein into the culture medium, and thus prokaryotic cells are employed. Suitable as host cells besides prokaryotes are also eukaryotic microbes which have been transfected with the expression vector. Thus, for example, it is possible to employ filamentous fungi or yeasts as suitable host cells for the vectors encoding the DNA sequences of the invention. Mention should be made in particular of Saccharomycis cirevesiae or the usual bakers' yeast product (Stinchcomb et al., Nature, 282:39, (1997)).

In a preferred embodiment, however, cells from multicellular organisms are chosen for the expression of DNA sequences of the invention. This also takes account of the possible need for glycosilation of the encoded proteins. This function can be carried out in a suitable way in higher eukaryotic cells—compared with prokaryotic cells. In principle, any higher eukaryotic cell culture is available as host cell, although cells of mammals, for example monkeys, rats, hamsters or humans, are very particularly preferred. The skilled worker is aware of a large number of established cell lines. In a list which is by no means definitive, the following cell lines are mentioned: 293T (embryonic kidney cell line), (Graham et al., J. Gen. Virol., 36:59 (1997)), BHK (baby hamster kidney cells), CHO (cells from hamster ovaries), (Urlaub and Chasin, P. N. A. S. (USA) 77:4216, (1980)), HeLa (human cervical carcinoma cells) and other cell lines.

For the purposes of the present invention, expression vectors having the DNA sequences of the invention are used to transfect preferably cells of the mammalian immune system, especially of the human immune system (claim 8).

A further aspect of the present invention are the gene products of the DNA sequences of the invention (claim 9). Gene products mean for the purposes of this invention both primary transcripts, i.e. RNA, preferably mRNA, and proteins or polypeptides, in particular in purified form (claim 10). These proteins have according to the invention at least one PYD domain and regulate or transport in particular inflammatory signals. The preferred purified gene product is one comprising one of the amino acid sequences (for a PYD domain) indicated in FIG. 7, including all functionally homologous alleles, fragments or derivatives. However, the proteins of the invention also include all proteins derived from DNA derivatives, DNA fragments or DNA alleles of the invention.

It is additionally possible for the proteins of the invention to be chemically modified. Thus, for example, a protective group may be present at the N terminus. Glycosyl groups may be attached to hydroxyl or amino groups, lipids (especially fatty acids, for example myristyl or palmityl acid) can be covalently linked to the protein of the invention, as can phosphates or acetyl groups and the like. It is also possible for any chemical substances, compounds or groups to be bound to the protein of the invention by any synthetic route. Additional amino acids, e.g. in the form of individual amino acids or in the form of peptides or in the form of protein domains and the like, can also be fused to the N and/or C terminus. In this case, so-called signal or leader sequences in particular are present at the N terminus of the amino acid sequence of the invention and guide the peptide cotranslationally or post-translationally into a particular cellular organelle or into the extracellular space (or the culture medium). It is also possible for amino acid sequences which, as antigen, permit binding of the amino acid sequence of the invention to antibody to be present at the N or at the C terminus. Mention should be made in this connection in particular of the Flag peptide whose sequence in the single-letter amino acid code is: DYKDDDDK or else His tag (at least 5, preferably at least 6 His residues). This sequence has strongly antigenic properties and thus permits rapid testing and easy purification of the recombinant protein. Monoclonal antibodies which bind the Flag peptide can be obtained from Eastman Kodak Co., Scientific Imaging Systems Division, New Haven, Conn. The DNA sequences of the invention may also be laid down in numerous exons which are separated from one another by introns on the strand of the genetic information molecule. This means that the invention relates also to all conceivable SPLICE variants (at the mRNA level) as gene products. The proteins encoded by these various SPLICE variants are also subject to this invention.

The present invention further relates to an antibody which recognizes an epitope on a gene product of the invention, in particular a protein of the invention (claim 12). The term “antibody” encompasses for the purposes of the present invention both polyclonal antibodies and monoclonal antibodies (claim 13), chimeric antibodies, anti-idiotype antibodies (directed against antibodies of the invention), all of which may be in bound or soluble form and, where appropriate, be marked by a label, as well as fragments of the aforementioned antibodies. Besides the fragments of antibodies of the invention on their own, it is also possible for antibodies of the invention to occur in recombinant form as fusion proteins with other (protein) components. Fragments as such or fragments of antibodies of the invention as components of fusion proteins are typically produced by the methods of enzymatic cleavage, of protein synthesis or the recombination methods familiar to the skilled worker.

The polyclonal antibodies are heterogeneous mixtures of antibody molecules which are produced from sera of animals which have been immunized with an antigen. A monoclonal antibody comprises an essentially homogeneous population of antibodies which are specifically directed against antigens, the antibodies essentially having identical epitope binding sites. Monoclonal antibodies can be obtained by processes known in the prior art (e.g. Kohler and Milstein, Nature, 256, 495-397, (1975); U.S. Pat. No. 4,376,110; Ausubel et al., Harlow and Lane “Antikorper”: Laboratory Manual, Cold Spring, Harbor Laboratory (1988)). The description contained in the aforementioned references is included as constituent of the present invention in the disclosure of the present invention. Antibodies of the invention may belong to one of the following immunoglobulin classes: IgG, IgM, IgE, IgA, GILD and, where appropriate, a subclass of the aforementioned classes. A hybridoma cell clone which produces monoclonal antibodies of the invention can be cultivated in vitro, in situ or in vivo. The production of large titers of monoclonal antibodies preferably takes place in vivo or in situ.

The chimeric antibodies of the invention are molecules which comprise various components which are derived from various species (e.g. antibodies having a variable region which is derived from a mouse monoclonal antibody, and a constant region of a human immunoglobulin). Chimeric antibodies are preferably employed in order, on the one hand, to reduce the immunogenicity on use, and on the other hand to increase the yields during production, e.g. murine monoclonal antibodies afford higher yields from hybridoma cell lines but also lead to higher immunogenicity in humans, so that human/murine chimeric antibodies are preferably employed. Chimeric antibodies and methods for producing them are known from the prior art (Cabilly et al., Proc. Natl. Sci. USA 81: 3273-3277 (1984); Morrison et al. Proc. Natl. Acad. Sci USA 81:6851-6855 (1984); Boulianne et al. Nature 312 643-646 (1984); Cabilly et al., EP-A-125023; Neuberger et al., Nature 314: 268-270 (1985); Taniguchi et al., EP-A-171496; Morrion et al., EP-A-173494; Neuberger et al., WO 86/015333; Kudo et al., EP-A-184187; Sahagan et al., J. Immunol. 137: 1066-1074 (1986); Robinson et al., WO 87/02671; Liu et al., Proc. Natl. Acad. Sci USA 84:3439-3443 (1987); Sun et al., Proc. Natl. Acad. Sci USA 84:214218 (1987); Better et al., Science 240: 1041-1043 (1988) and Harlow and Lane, Antikörper: A Laboratory Manual, as cited above. These citations are included in the present invention as belonging to the disclosure.

Such an antibody of the invention is very particularly preferably directed against a sequence section on the PYD domain as epitope (claim 14).

An anti-idiotype antibody of the invention is an antibody which recognizes a determinant which is generally associated with the antigen-binding site of an antibody of the invention. An anti-idiotype antibody can be produced by immunizing an animal of the same species and of the same genetic type (e.g. of a mouse strain) as starting point for a monoclonal antibody against which an anti-idiotype antibody of the invention is directed. The immunized animal will recognize the idiotypic determinants of the immunizing antibody through the production of an antibody which is directed against the idiotypic determinants (namely an anti-idiotic antibody of the invention) (U.S. Pat. No. 4,699,880). An anti-idiotype antibody of the invention can also be employed as immunogen in order to elicit an immune response in another animal and in order to lead to production therein of a so-called anti-anti-idiotype antibody. The anti-anti-idiotype antibody may, but need not, be identical in terms of its epitope construction with the original monoclonal antibody which elicited the anti-idiotypic response. It is possible in this way through the use of antibodies directed against idiotypic determinants of a monoclonal antibody to identify other clones which express antibodies of identical specificity.

Monoclonal antibodies directed against proteins of the invention, analogs, fragments or derivatives of these proteins of the invention can be employed to induce the binding of anti-idiotype antibodies in appropriate animals such as, for example, the BALB/c mouse. Cells from the spleen of such an immunized mouse can be used to produce anti-idiotype hybridoma cell lines which secrete anti-idiotype monoclonal antibodies. A further possibility is for anti-idiotype monoclonal antibodies also to be coupled to a carrier (KLH, “keyhole limpet hemocyanin”) and then be used to immunize further BALB/c mice. The sera of these mice then contain anti-anti-idiotype antibodies which have the binding properties of the original monoclonal antibodies and are specific for an epitope of the protein of the invention or of a fragment or derivative of the same. The anti-idiotype monoclonal antibodies have in this way their own idiotypic epitopes or “idiotopes”, which are structurally similar to the epitope to be investigated.

The term “antibody” is intended to include both intact molecules and fragments thereof, e.g. Fab and F(ab′)₂. Fab and F(ab′)₂ fragments lack an Fc fragment as present for example in an intact antibody, so that they can be transported more quickly in the blood stream and display comparatively less nonspecific tissue binding than intact antibodies. It is emphasized in this connection that Fab and F(ab′)₂ fragments of antibodies of the invention can be employed in the detection and quantification of proteins of the invention. Such fragments are typically produced by proteolytic cleavage using enzymes such as, for example, papain (to produce Fab fragments) or pepsin (to produce F(ab′)₂ fragments).

Antibodies of the invention, including the fragments of these antibodies, can be employed for the quantitative or qualitative detection of protein of the invention in a sample, or else for the detection of cells which express and, where appropriate, secrete proteins of the invention. Detection can be achieved with the aid of immunofluorescence methods which are carried out fluorescence-labeled antibodies in combination with light microscopy, flow cytometry or fluorometric detection.

Antibodies of the invention (or fragments of these antibodies) are suitable for histological investigations such as, for example, within the framework of immunofluorescence or immunoelectro-microscopy, for the in situ detection of a protein of the invention. The in situ detection can take place by taking a histological sample from a patient and adding labeled antibodies of the invention to such a sample. The antibody (or a fragment of this antibody) is applied in labeled form to the biological sample. It is possible in this way to determine not only the presence of protein of the invention in the sample but also the distribution of the protein of the invention in the investigated tissue. The biological sample may be a biological fluid, a tissue extract, harvested cells such as, for example, immune cells or myocardial or liver cells, or generally cells which have been incubated in a tissue culture. The labeled antibody can be detected depending on the nature of the label by methods known in the prior art (e.g. by fluorescence methods). The biological sample may, however, also be applied to a solid-phase carrier such as, for example, nitrocellulose or another carrier material, so that the cells, cell parts or soluble proteins are immobilized. The carrier can then be washed one or more times with a suitable buffer, subsequently treating with a detectably labeled antibody of the present invention. The solid-phase carrier can then be washed with the buffer a second time in order to remove unbound antibodies. The amount of bound label on the solid-phase carrier can then be determined by a conventional method.

Particularly suitable carriers are glass, polystyrene, polypropylene, polyethylene, dextran, nylon-amylases, natural or modified celluloses, polyacrylamides and magnetite. The carrier may be either of limited solubility or insoluble in nature in order to satisfy the conditions specified in the present invention. The carrier material may assume any shapes, e.g. be in the form of beads, or cylindrical or spherical, with polystyrene beads being preferred as carrier.

A detectable antibody labeling can take place in various ways. For example, the antibody can be bound to an enzyme, in which case the enzyme can eventually be employed in an immunoassay (EIA). The enzyme may then react later with an appropriate substrate to result in a chemical compound which can be detected and, where appropriate, quantified in a manner familiar to a skilled worker, e.g. by spectrophotometry, fluorometry or other optical methods. The enzyme may be malate dehydrogenase, staphylococcal nuclease, delta-5-steroid isomerase, yeast alcohol dehydrogenase, alpha-glycerophosphate dehydrogenase, triosephosphate iso-merase, horse raddish peroxidase, alkaline phsophatase, aspariginase, glucose oxidase, beta-galactosidase, ribonuclease, urease, catalase, glucose-6-phosphate dehydrogenase, glucoamylase or acetylcholinesterase. Detection is then made possible via a chromogenic substrate which is specific for the enzyme employed for the labeling, and can eventually take place for example via visual comparison of the substrate converted by the enzymic reaction compared with control standards.

Detection may furthermore be ensured by other immunoassays, e.g. by radioactive labeling of the antibodies or antibody fragments (i.e. by a radio-immunoassay (RIA; Laboratory Techniques and Biochemistry in Molecular Biology, Work, T. et al. North Holland Publishing Company, New York (1978). The radioactive isotope can in this case be detected and quantified by use of scintillation counters or by autoradigraphy.

Fluorescent compounds can likewise be employed for the labeling, for example compounds such as fluorescin isothiocyanate, rhodamine, phyoerythrin, phycocyanin, allophycocyanin, o-phthaldehyde and fluorescamine. It is also possible to employ fluorescence-emitting metals such as, for example, ¹⁵²E or other metals from the lanthanide group. These metals are coupled to the antibody via chelating groups such as, for example, diethylenetriaminepentaacetic acid (ETPA) or EDTA. A further possibility is for the antibodies of the invention to be coupled via a compound which acts by means of chemiluminescence. The presence of the chemiluminescence-labeled antibody is then detected via the luminescence which appears during a chemical reaction. Examples of such compounds are luminol, isoluminol, acridinium ester, imidazole, acridinium salt or oxalate ester. It is equally possible also to employ bioluminescent compounds. Bioluminescence is a subtype of chemiluminescence which is found in biological systems where a catalytic protein enhances the efficiency of the chemiluminescent reaction. The bioluminescent protein is again detected via the luminescence, an example of a suitable bioluminescent compound being luciferin, luciferase or aequorin.

An antibody of the invention can be applied for use in an immunometric assay, also known as two-site or sandwich assay. Typical immunometric assay systems include so-called “forward” assays which are distinguished by antibodies of the invention being bound to a solid-phase system and by the antibody being brought into contact in this way with the sample which is investigated. In this way, the antigen from the sample is isolated from the sample through the formation of a binary solid-phase antibody-antigen complex. After a suitable incubation time, the solid carrier is washed in order to remove the remaining residue of the liquid sample, including the antigen which is unbound where appropriate, and then brought into contact with a solution which contains an unknown quantity of labeled detection antibody. The labeled antibody serves in this case as so-called reporter molecule. After a second incubation time which permits the labeled antibody to associate with the antigen bound to the solid phase, the solid-phase carrier is again washed in order to remove labeled antibodies which have not reacted.

In an alternative assay form it is also possible to employ a so-called sandwich assay. In this case, a single incubation step may suffice if the antibody bound to the solid phase and the labeled antibody are both applied simultaneously to the sample to be tested. After completion of the incubation, the solid-phase carrier is washed in order to remove residues of the liquid sample and of the unassociated labeled antibodies. The presence of labeled antibody on the solid-phase carrier is determined in exactly the same way as in the conventional “forward” sandwich assay. In the so-called reverse assay there is stepwise addition firstly of a solution of the labeled antibody to the liquid sample, followed by admixture of unlabeled antibody bound to a solid-phase carrier after a suitable incubation time has elapsed. After a second incubation step, the solid-phase carrier is washed in a conventional way in order to free it of sample residues and of labeled antibody which has not reacted. Determination of the labeled antibody which has reacted with the solid-phase carrier is then carried out as described above.

A further aspect of the present invention is a method for isolating gene products having at least one PYD domain, where the host cells are transformed with an expression vector of the invention and then cultivated under suitable conditions promoting expression, so that the gene product can eventually be purified from the culture (claim 15). The protein of the DNA sequence of the invention can in this case be isolated from a culture medium or from cell extracts, depending on the expression system. The skilled worker is able to appreciate directly that the particular isolation methods and the method for the purification of the recombinant protein encoded by a DNA of the invention depends greatly on the type of host cell or else on whether the protein is secreted into the medium. For example, it is possible to employ expression systems which lead to secretion of the recombinant protein. The culture medium must in this case be concentrated by commercially obtainable protein-concentration filters, e.g. Amicon or Millipore Pelicon. The concentration step can be followed by a purification step, e.g. a gel filtration step. However, an alternative possibility is also to employ an anion exchanger which has a matrix with DEAE.

All materials known from protein purification, e.g. acrylamide or agarose or dextran or the like, are suitable as matrix in this case. However, it is also possible to employ a cation exchanger, which then typically contains carboxymethyl groups. HPLC steps can then be used for further purification of a protein encoded by a DNA of the invention. One or more steps may be involved. In particular, the reversed phase method is employed. These steps serve to obtain an essentially homogeneous recombinant protein of a DNA sequence of the invention.

Besides bacterial cell cultures for isolating the gene product, it is also possible to employ transformed yeast cells. The translated protein can be secreted in this case, so that protein purification is simplified. Secreted recombinant protein from a yeast host cell can be obtained by methods like those disclosed by Urdal et al. (J. Chromato. 296:171 (1994)).

A further aspect of the present invention is a method for the expression of gene products having at least one PYD domain, where host cells are transformed with an expression vector which comprises a DNA sequence of the invention (claim 17). This method for the expression of gene products based on a DNA sequence of the invention is used not to concentrate and purify the corresponding gene product, but on the contrary to influence cellular metabolism through the introduction of the DNA sequences of the invention via the expression of the relevant gene product. In this case, consideration should be given in particular to the use of the host cells which have been transformed with the aid of expression vectors for the purpose of eliminating the inflammatory response. It is possible through the use of a so-called constitutive promoter to express in these cells constant concentrations of proteins based on sequences of the invention. Initiation of inflammation is suppressed permanently in this way.

The corresponding cell lines thus become resistant to a large number of inflammatory stimulants. These modified cells can also, where appropriate, be transferred back into the mammalian or human organism again. In this way, use of the DNA sequences of the invention for gene therapy becomes possible through cells manipulated in vitro with the expression vectors of the invention and the subsequent transfer (transplantation) into the organism. In this case, expression vectors having the sequences of the invention are preferably transfected into cells which succumb to dysregulation of the inflammation in the organism (for example hepatocytes during chronic hepatitis). Sequences of the invention particularly employed in such a gene therapy approach are those which block the inflammatory signal, for example fragments which do not transmit the inflammatory signal and thus interrupt signal transduction.

However, the present inventive concept is also associated with a gene therapy method which can be carried out in vivo. Employed for this purpose are vectors (e.g. liposomes, adenoviruses, retroviruses or the like or else naked DNA) which insert the DNA sequences of the invention specifically into the desired target cells of the organism. The target cells are typically cells whose regulation of inflammation is impaired, in particular cells which pathologically show an enhanced disposition to the inflammatory response (for example in chronic hepatitis). It is possible to employ in this connection fragments of a DNA sequence of the invention which display an inhibitory effect, for example DNA sequences which comprise essentially only the PYD domain and thus are now able only to carry out an association function but not able to transmit further biological signals (for the inflammation)—i.e. display no further biological functionality.

The present invention further relates to the use of a DNA sequence of the invention (alleles, derivatives, fragments) or of a gene product of the invention for the treatment of disorders based on dysregulation of intracellular signal transduction (claim 17). The aforementioned use according to the invention of DNA sequences also includes the use of expression vectors of the invention described above, which have a nucleotide sequence of the invention, for example a nucleotide sequence disclosed in FIG. 1 or a functional derivative or allele of such a sequence (or of an infunctional derivative of such a sequence), with the aim of correcting the misdirected intracellular signal transduction. The use can take place by gene therapy methods via injection of naked DNA of the invention or the protein or via gene ferries, in particular viral or liposomal vectors. The present invention therefore relates both to the use of such expression vectors of the invention and to the use according to the invention of cells (i.e. the use for inducing (or for blocking) the inflammatory event) which are transfected with expression vectors of the invention.

It can be stated specifically that such gene therapy approaches have already proved to be effective in the therapy of genetically related disorder, for example in hemophilia, and also to be effective in the treatment of other genetic disorder such as, for example, cystic fibrosis. In a preferred embodiment, a nucleotide sequence of the type according to the invention, for example a nucleotide sequence according to the nucleotide sequences present in FIG. 1 or a variant (derivative, fragment (in particular comprise at least 100 bases, or allele) of this sequence is transferred into a suitable vector which inserts the nucleic acid sequence of the invention into the mammalian cells, preferably human cells. Particularly suitable transfection vectors for this application are retroviruses and adenoviruses. An alternative possibility is also for nucleic acid sequences of the invention to be complexed in a molecular conjugate with a virus (for example an adenovirus) or with viral components (for example capsid proteins). Suitable methods for forming such vectors are well known in the prior art, reference being made for example to the disclosure in “Working toward Human Genetherapy”, chapter 28 (in Recombinant DNA, Second Edition, Watson GD et al, New York: Scientific American Books, p. 567-581, 1992). In such a gene therapy method, vectors transfected with nucleotide sequences of the invention are administered to the affected patient in cells or tissues preferably by injection, inhalation, oral intake or intake through mucous membranes. Such a trial approach is generally referred to as in vitro gene therapy. Alternatively, cells or tissues, for example hematopoietic (stem) cells from the bone marrow or other adult stem cells (especially tissue-specific stem cells) can be taken from the affected patient and cultivated in vitro as specified in the methods known in the prior art. The correspondingly configured vectors with the nucleotide sequences of the invention are then added in vitro to the cells or tissues, with such vectors being incorporated into the cells preferably by electroporation. The cells or tissues modified in this way are finally reimplanted into the affected patient. Such gene therapy methods are referred to as ex vivo gene therapy. For both gene therapy approaches, i.e. in vivo or ex vivo methods, it is possible for nucleotide sequences of the invention to be operatively linked to a regulatory DNA sequence, in which case this may also be a heterologous regulatory DNA sequence, so that a recombinant construct is present in the transfected cell. This construct can then be inserted into the vector and finally administered directly to the affected patient in an in vivo gene therapy approach or to the cells or tissues of the affected patient in an ex vivo gene therapy approach. It is possible in a further preferred embodiment for the genetic construct to be administered to the cells or the tissues of the animal either in vivo or ex vivo in a molecular conjugate with a virus (for example an adenovirus or viral components (for example viral capsid proteins). The gene therapy approaches described above may lead either (a) to a homologous recombination between the nucleotide sequence and the infunctional gene in the cells of the affected animal or (b) to a random insertion of the gene at any site in the host cell genome or (c) to incorporation of the gene into the cell nucleus, in which case it may then be present as extrachromosomal genetic element. The disclosure of such methods and approaches for the gene therapy can be found in the US patent U.S. Pat. No. 5,578,461, WO 94/12650 and WO 93/09222, which form part of the disclosure of the present application. The transfected host cells, which may be homologous or heterologous, can be enveloped with a semipermeable layer and be reimplanted in this way into the affected patient, in this way preventing the patient's immune system attacking the reimplanted cells (see WO 93/09222).

It is preferred in this connection to use a DNA sequence of the invention or a gene product of the invention when the disorder is one with an excessive inflammatory response (claim 18). It is very particularly preferred to use such a DNA sequence or such a gene product for the treatment (for producing a drug product for the treatment) of psoriasis, arteriosclerosis, bacterial or viral infectious diseases, especially bacterial or viral meningitis or bacterial pneumonia, multiple sclerosis, rheumatoid arthritis, asthma, sarcoidosis, glomerular nephritis or osteoarthritis (claim 19). This can be achieved for example by fragments which block signal transduction, for example sequences which code only for the PYD domain (or parts thereof) or comprise only the latter.

In a further aspect of the present invention there is provision of a compound which is characterized in that it inhibits the function of the gene products (proteins) of the invention as intracellular signal molecule of an inflammatory signal cascade to induce inflammatory responses. In particular, compounds of the invention block the specific interaction of PYD domains for intracellular signal transmission (claim 20). A chemical compound of the invention which blocks the PYD function of the invention (for example in inflammation or apoptosis) is preferably an oligopeptide which may be chemically modified (for example to facilitate passage through the cell membrane, in particular by terminal (especially N terminal) sequence regions) or may be unmodified. It may in particular be a native sequence region which is involved in PYD association of a protein of the invention. Mention may be made for example of so-called DN variants (for example AA 1 to 94 of human Pycard or corresponding PYD domains of NALP proteins (see FIG. 1)) which can be administered for example also the gene therapy methods described below. Such DN variants may also comprise exclusively the NAD domain or a part thereof or one the LRR domain or a part thereof or a CARD domain or a part thereof and, in this way, cause dominant negative blocking of the signal cascade, in particular inflammatory signal cascades. The medical indications for use (production of drug products) of such DN variants of native sequences of the invention are disclosed hereinafter. Thus, sequences of individual domains (LRR, PYD, CARD, NAD) of sequences of the invention are disclosed according to the invention.

It is thus possible for example for an inhibitory molecule of the invention, preferably a tetra- to dodecamer, to comprise an amino acid sequence corresponding to the native substrate or to consist of a native substrate sequence, with the preferably tetra- to dodecamer typically also having a sequence section from a PYD domain of a protein of the invention. It is possible where appropriate for such an oligopeptide also to be chemically modified by replacing the amide-like linkage between the individual amino acids by an alternative chemical group (for example sulfur or phosphorus bridges) which is resistant to proteolytic degradation.

A chemical compound of the invention is preferably an organic chemical compound with a molecular weight of <5000, in particular <3000, especially <1500 and is typically physiologically well tolerated (claim 21). It will where appropriate be a component of a composition having at least one other active substance and, preferably, excipients and/or additives and be able to be employed as drug product. The organic molecule will be particularly preferred when the binding constant for binding to a protein of the invention, in particular to the PYD domain of a protein of the invention, is at least 10⁷ mol⁻¹. The compound of the invention will preferably be constituted in such a way that it can cross the cell membrane, whether by diffusion or via (intra)membrane transport proteins (claim 22).

In another preferred embodiment of the present invention, the compound of the invention is an antibody, preferably an antibody which is directed against the PYD domain of a protein of the invention and which is inserted ex vivo into retransplanted host cells or by gene therapeutic in vivo methods into host cells and there is not, as “intrabody”, secreted but is able to display its effect intracellularly. The cells are protected from an inflammatory response by such “intrabodies” of the invention. Such a procedure will typically be suitable for cells of those tissues in the patient which show a pathophysiologically excessive inflammatory behavior, i.e. for example in hepatocytes (hepatitis), keratinocytes, connective tissue cells, immune cells or muscle cells. Accordingly, cells modified by gene therapy in such a way with “intrabodies” of the invention also form part of the present invention.

Specifically, antibodies of the invention against amino acid sequences of the invention, especially antibodies of the invention which are directed against a PYD domain, and especially when they are in the form of single-chain Fv (scfv) or Fab fragments and can be guided into various subcellular compartments for recognition of the target structure (for example of the PYD domain) are suitable for blocking the activity of the target molecule therein, either directly or indirectly by interference with the subcellular transport pathways. For example, it is possible for targeted subcellular positioning of the intrabody to attach an ER retention signal (KDEL) to the C terminus of the antibody fragment with a so-called leader sequence, which leads to retention in the lumen of the endoplasmic reticulum. Transport into the mitochondria can be achieved by a corresponding mitochondrial leader sequence, for example of the cytochrome C oxidase unit VIII. Cytoplasmic expression of the antibody is ensured by the expression of the antibody fragments taking place without any signal or leader sequence. Transport into the nucleus is also possible by, for example, choosing a nuclear localization sequence (PKKKRKV) from the large SV 40 T antigen, specifically either at the N or C terminus. Appropriate technical measures must be employed in order to ensure expression in the reducing milieu of the cytoplasm with formation of disulfide bridges. The following publications from the prior art are explicitly included in the context of the present disclosure (Marasco, 1997, Gene Therapy. 4, 11-15; Richardson, & Marasco, 1995, Trends Biotechnology 13, 306-310; Biocca and Cattaneo 1995, Trends Cell Biology 5, 248-252; Biocca et al, 1995, Bio/Techn. 13, 1110-1115, Biocca et al, 1990, EMBO Journal 9, 101-108; Piche et al, 1998, Cancer Research 58, 2134-2140; Rosso et al, 1996, Biochem. Biophys. Res. Communication 220, 255 to 263).

Further suitable and preferred compounds of the invention (for example for inhibiting molecular mechanisms which cause excessive inflammatory events) are also sequences (DNA or RNA) which are associated with antisense technologies. In this case, antisense DNA or RNA are inserted into cells (for example by gene therapeutic approaches, for example use of recombinant viruses, see above) and these can in this way, by complementary binding of transcribed mRNA (for proteins of the invention containing PYD domains) block the translation of the polymorphic genomic sequence belonging thereto. Such a procedure is relevant in particular for patients whose expression level of proteins containing PYD domains is pathologically increased.

Further compounds and therapeutic methods for the treatment of the disclosed medical indications (and for producing a drug product) are based on ribozyme methods. The ribozymes used in this case are able to cut a target mRNA. In the present case, therefore, ribozymes which are able to cleave native mRNA of proteins of the invention (for example NALP1 or other proteins containing PYD domains) are disclosed. Ribozymes of the invention must moreover be able to interact with the target mRNA of the invention, for example by base pairing, and subsequently cleave the mRNA in order to block the translation of, for example, NALP1 or Pycard. The ribozymes of the invention are introduced into the target cells via suitable vectors (in particular plasmids, modified animal viruses, especially retroviruses), the vectors having a cDNA sequence for a ribozyme of the invention besides, where appropriate, other sequences.

A chemical compound of the invention having the function of blocking the, for example, inflammatory function of physiological proteins of the invention (see FIG. 1) can be used as drug product. In particular, a chemical compound of the invention is suitable (for producing a drug product) for the treatment of disorders for which at least partly a pathological hyperinflammatory response is causative or symptomatic. It is thus possible to employ an inhibitor according to the invention of the cellular function of a protein of the invention, i.e. for example of the inflammatory response, in particular an inhibitor of the association of PYD domains, for inhibition of inflammation very particularly in the treatment of the following disorders and for producing a drug product for the treatment of the following disorders: autoimmune diseases, psoriasis, arteriosclerosis, bacterial or viral infectious diseases, especially bacterial or viral meningitis or bacterial pneumonia, multiple sclerosis, rheumatoid arthritis, asthma, sarcoidosis, glomerular nephritis or osteoarthritis, Alzheimer's disease or Parkinson's disease (claim 23).

The present invention further relates to methods (“screening methods”) for identifying compounds (organic chemical compounds, biomolecules (for example oligonucleotides, antibodies or antibody fragments, oligopeptides, ribozymes, DN mutants of proteins of the invention) having inhibitory properties in relation to the induction or transmission of signals associated with inflammatory responses, especially of compounds which block the interaction of proteins of the inflammatory signal cascade, in particular of PYD domains (of identical or different proteins) which react together physiologically (for example block the association of the inflammosome by, for example, inhibiting the association of NALP1 and Pycard).

Other points according to the invention for attacking the interaction are, for example, inhibition of the interaction between the CARD domain of caspase-1 and the CARD domain of Pycard or inhibition of the interaction of the CARD domain of caspase-5 and NALPI. Finally, it may also be preferred for compounds of the invention to modulate (block or activate) the interaction of the LRR domain of proteins of the NALP class, for example NALP1, with proteins or stimuli located further upstream. It is also possible where appropriate for compounds which act as activators of the aforementioned interactions to be preferred.

Methods of the invention provide for (a) cells, especially hepatocytes, in particular T lymphocytes, being modified in such a way that they show an inflammatory response, (b) these cells modified according to (a) being provided in a cell culture, (c) test substances being added to the cell culture, (d) the the extent of the inflammatory response of the cells in the cell culture being determined. For this purpose, in the method of the invention preferably a plurality of parallel tests with increasing concentrations of the test substance are set up in order to be able, in the event of the test substance having an inhibitory effect on the inflammation, to determine the ID₅₀ thereof.

Alternatively, a method of the invention for identifying the aforementioned compounds may also encompass the following steps: (a) an in vitro test system which comprises at least one DNA sequence of the invention is provided, (b) in the form of a high-throughput screening, potential active substances are added to the in vitro test system provided according to (a), and (c) a physical, chemical or biological signal in the test system is detected to identify active substances. Chemical libraries can be screened, in particular both for activating or inhibiting substances. In this connection, reference is made to the disclosure of the textbook by Böhm, Klebe and Kubinyi (Wirkstoffdesign, 1996, Spektrum-Verlag, Heidelberg), the contents of which in this regard being incorporated herein by reference. In particular, compounds identified [lacuna] one of the aforementioned methods of the invention may also be suitable for influencing the expression level of the native sequences of the invention, for example of Pycard or NALP1. The mechanism of action may be based on influencing for example the activity of the native promoter, so that the transcription activity is modulated.

A screening method of the invention may also be carried out with the aid of so-called proteomics techniques. For this purpose, to determine a standard, typical differences in the expression pattern of cells with inflammatory response and control cells are found experimentally. The method typically employed for such a method is 2D gel electrophoresis. Test substances which alter the expression pattern of substances of the invention can then be identified on the basis of the altered expression patterns (see also the disclosure in Rehm, H., Der Experimentator, Spektrum-Verlag, 2000).

It is additionally possible by structural analyses of a protein of the invention specifically to find compounds of the invention which have a specific binding affinity (rational drug design (Bohm, Klebe, Kubinyi, 1996, Wirkstoffdesign, Spektrum-Verlag, Heidelberg)). In this case, the structure or a partial structure, derivative, allele, isoform or a part of such of one of the proteins of the invention is established by NMR or X-ray crystallographic methods (after appropriate crystallization, e.g. by the “hanging drop” method) or, if such a highly resolved structure is not available, a structural model of a protein of the invention is produced with the aid of structure-prediction algorithms, for example also with the aid of homologous proteins whose structure has already been elucidated (e.g. of rhodopsin), and the latter is (are) used to identify, with the assistance of molecular modeling programs, compounds which can act as agonists or antagonists and for which a high affinity for the protein of the invention can be predicted. It is possible where appropriate for the methods defined above also to be combined together for the structural elucidation. Suitable force fields are employed to simulate the affinity of a compound potentially having affinity to a substructure of interest of a protein of the invention, for example the active site, a binding cavity or a hinge region. These substances are then synthesized and tested for their binding capacity and their therapeutic utilizability in suitable test methods. Such in silico methods for identifying potential active substances which display their effect by binding to PYD domain proteins of the invention are likewise an aspect of the present invention.

Also disclosed in addition are, in particular, in vitro methods which permit naturally occurring polymorphisms of the proteins of the invention to be identified. Particularly suitable for such purposes of use are PCR methods, especially also RT-PCR methods, i.e. diagnosis on the basis of mRNA which is appropriately translated in vitro into cDNA and then amplified with the aid of conventional PCR methods. Appropriate array techniques which position oligonucleotides of the invention on a chip also permit diagnosis with the aid of hybridization reactions. In this case, the patient's sample is tested against an array with oligonucleotides of the invention which include the polymorphisms of the invention. Positive signals on the array with oligonucleotides which show polymorphisms coupled to inflammatory disorders allow a corresponding diagnosis to be made.

Finally, the present invention relates to oligopeptides which [lacuna] partial sections comprising at least 20, more preferably at least 30 and even more preferably at least 50 amino acids of the protein sequences disclosed in FIG. 1, especially sequences with the numbers 1.2, 1.3, 1.4, 1.5, 1.6, . . . , 1.20. Such partial sequences can be for example chemically synthesized by methods familiar to the skilled worker and can preferably be employed as antigens for producing antibodies. These partial sections of proteins of the invention or derivatives, alleles or fragments thereof will preferably [lacuna] disclosed sequences which, in the three-dimensional model of the proteins, occupy those regions which account for at least part of the protein surface. Preferred partial sequences with a length of at least 20 AA will include at least in part the PYD domain (see FIG. 7) (or in the case of the proteins of the NALP protein class the NAD domain) of the proteins of the invention, and a partial section of the invention will particularly preferably have peptides with a length of at least 20 AA of one of the sequences of the invention as shown in FIG. 7 between position 1 and position 30 (as shown in FIG. 7), for example the peptide LENLPAEELKKFKLKLLSVPL (position 1 to 21, 21 AA length of human Pycard) or the corresponding sequences to be found in FIG. 7 of the other sequences of the invention, in particular from the NALP protein class.

The present invention is explained in more detail by the following figures:

FIG. 1 represents DNA sequences (including the corresponding amino acid sequences) which are involved in the inflammatory signal cascade.

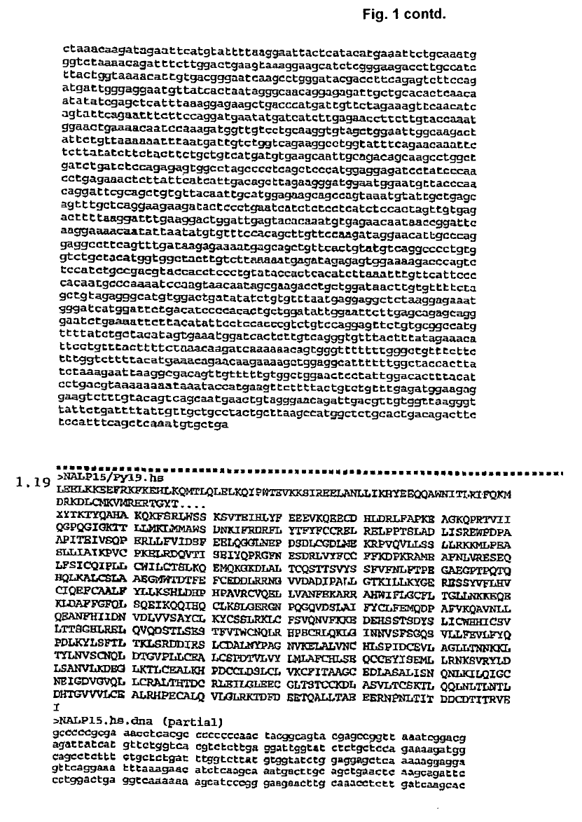

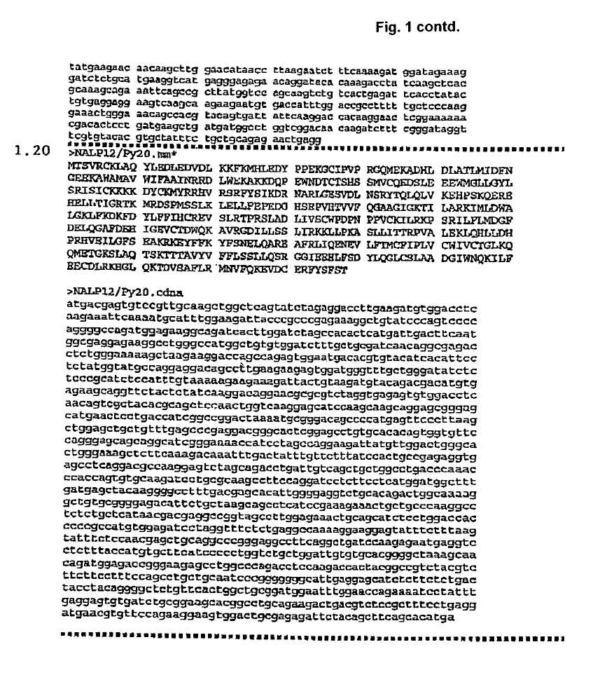

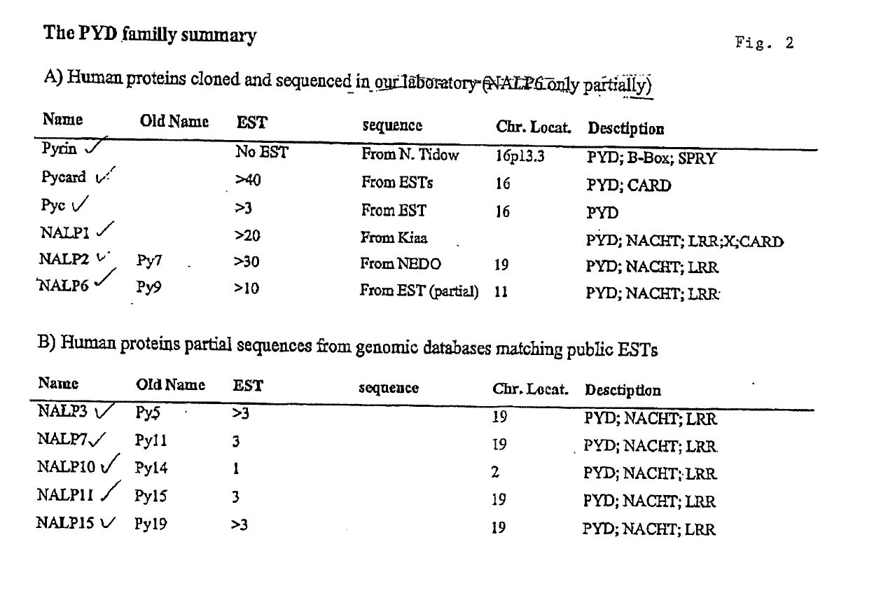

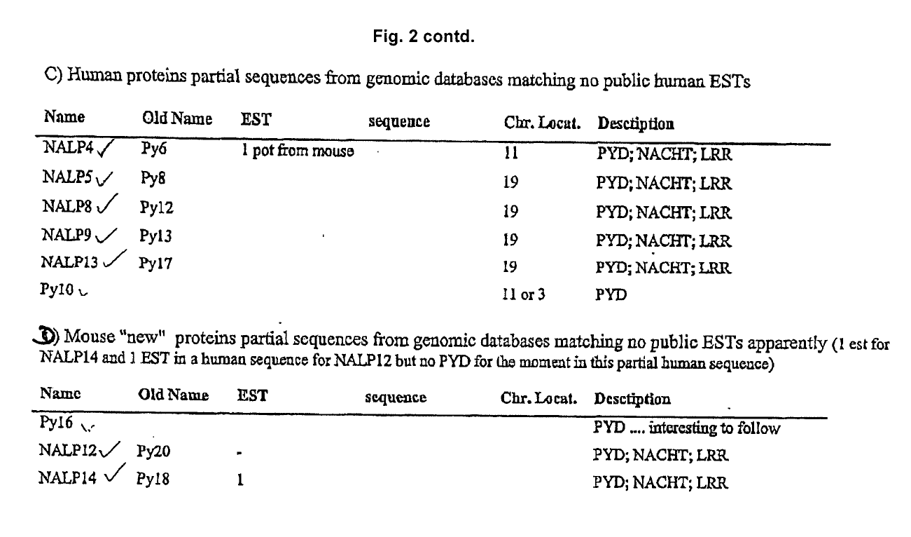

FIG. 2 shows a summary of the aforementioned sequences in tabular form arranged according to the designations already used in FIG. 1, indicating any EST clones, the origin of the sequence, the location on a chromosome, and a summary of the domains present in the respective sequences (e.g. the PYD domain, SPRY domain, CARD domain, NACHT domain, the LRR domain). The sequences of human origin are summarized under (A), (B) and (C), whereas (D) contains murine sequences.

FIG. 3 shows the generalized “PYD” search profile which was employed to find further PYD proteins in, for example, EST databases. Reference is made in this connection to the publication by Bucher et al. (1996, Computer Chem., 20, 3-24) and to the corresponding explanations in the published German patent application DE 197 13 393.2-41, both of which to this extent form part of the present application. In this way, two further PYD-containing proteins were identified, namely NALP1 and NALP2 (the designation NALP is composed of the individual characteristic domains occurring in both proteins: NACHT, LRR and PYD domain). Both proteins play a crucial part as signal transduction proteins for inflammatory signals. In addition, their function and thus use in connection with apoptotic signal protein transduction also comes under consideration.

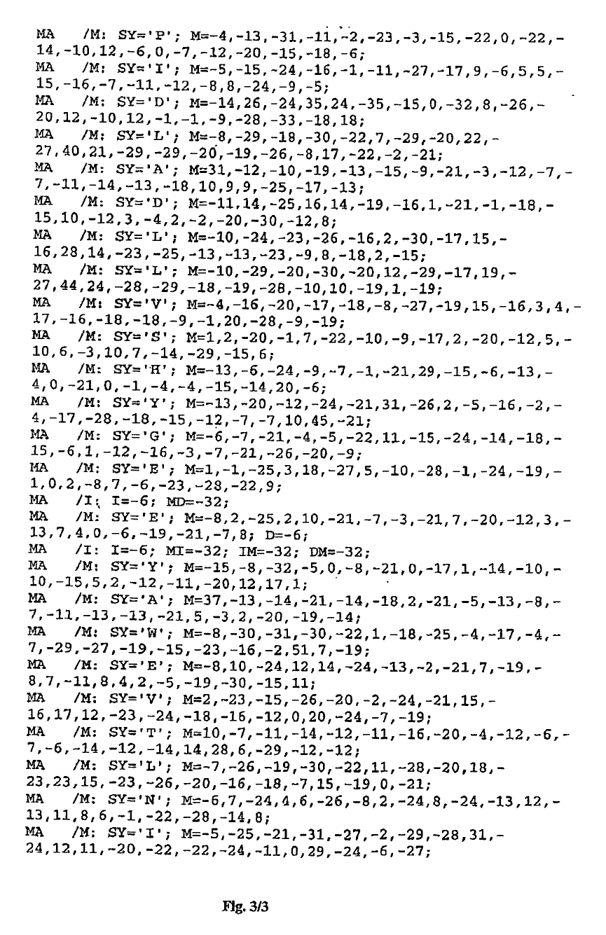

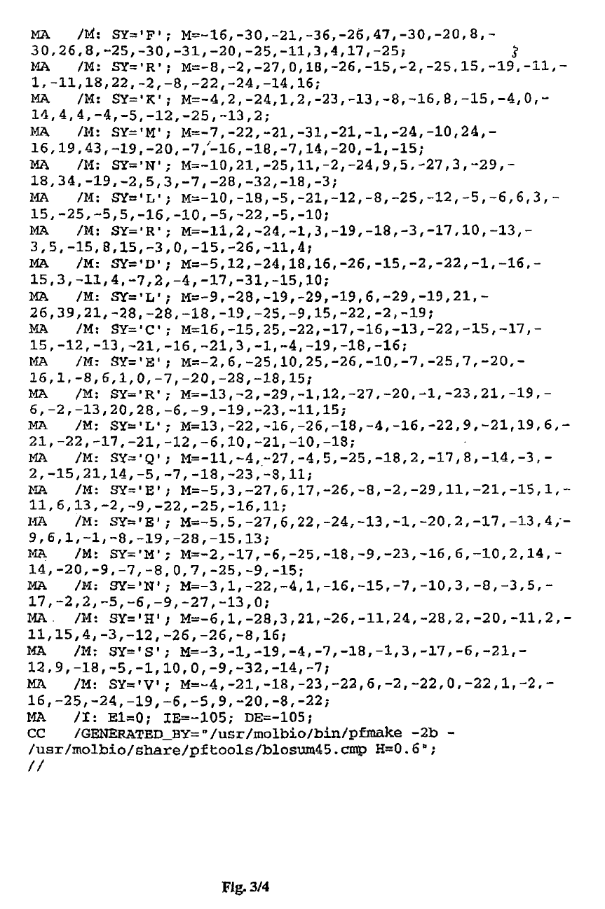

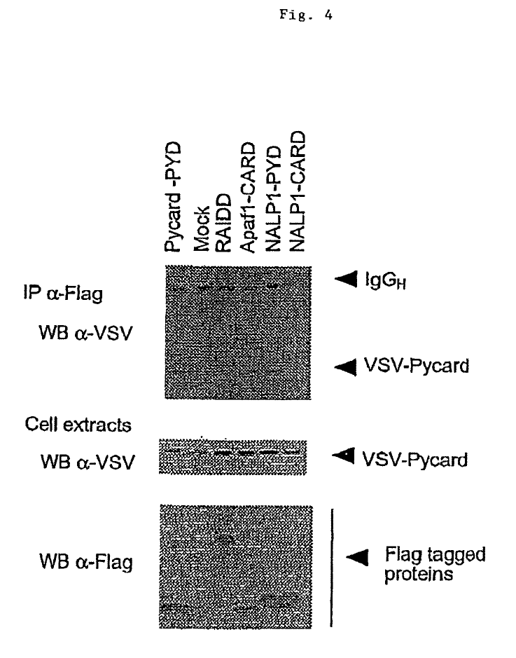

Overall, FIG. 4 shows that Pycard homodimerizes with the aid of its PYD domain and interacts with the PYD domain of NALP1. In the central depiction, cell extracts, lysed 24 hours after the transfection, were used and the anti-Flag immunoprecipitates were investigated for the presence of VSV-Pycard. In the lower depiction, the various Flag-labeled constructs (namely Pycard PYD (i.e. PYD from Pycard)), RAIDD, Apafl CARD (CARD from Apafl, NALP1 PYD (PYD from NALP1), NALP1 CARD (CARD from NALP1) and a mock vector were investigated for their presence in each case in the immunoprecipitate by anti-Flag antibodies.

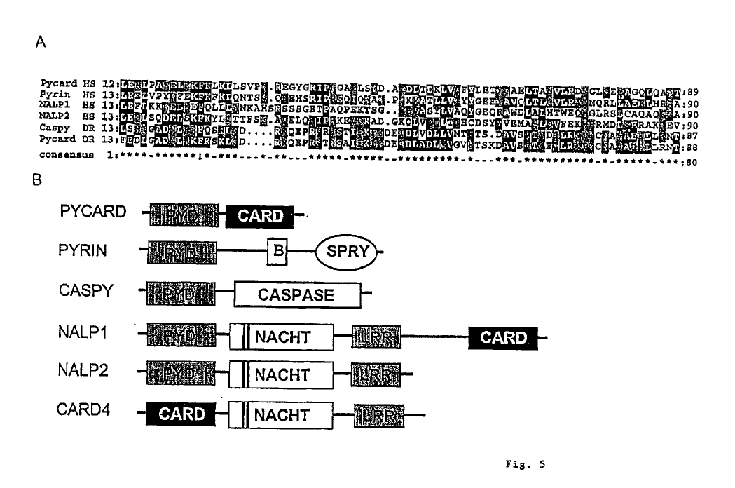

FIG. 5 shows in part an alignment of PYD domains from various proteins (alignment from N terminus to C terminus). The respective sequence positions with at least 50% identity and with at least similar amino acids are respectively given black and gray backgrounds. The abbreviations stand for HS: homo sapiens and DR: Danio rerio (zebra fish). The respective access numbers in the “Gen Bank” (EMBL) database are as follows: AF310103 for human Pycard, AF310104 for murine Pycard, 015553 for Pyrin, AF310105 for NALP1, AF310106 for NALP2, AAF66964 for the zebra fish CASPY protein, AAF66956 for the zebra fish Pycard protein.

FIG. 5B represents in a diagrammatic manner the domain structure of proteins having a PYD domain (Pyrin domain). The naming of the individual homology domains is to be found in the figure legend in annex A12 under FIG. 5.

FIG. 5C represents the amino acid sequence of NALP1. The various shadings correspond to the domain-specific shadings chosen for FIG. 5B: dark gray background: PYD domain, framed pale background NACHT domain, pale gray background: LRR domain and dark background: CARD domain.

FIG. 6 represents an alignment of representative Pyrin domains (PYD domain) death effector domain (DED) and caspase recruitment domain (CARD) and death domain (DD). The similarity of the various domains becomes clearer through corresponding amino acids, which are depicted with shading. There are considerable similarities also at the level of the secondary structure, as is clear through the secondary structure prediction (for 6 α-helices in each case here).

FIG. 7 represents an alignment (from the N to the C terminus) of amino acid sequences of the PYD domains of the following proteins: Pyrin, human (hs), mouse (mm) and rn, human and murine Pycard human Pyc, human NALP1, human NALP2, human NALP3, human NALP4, human NALP5, human NALP6, human NALP7, human NALP8, human NALP9, human NALP10, human NALP11, murine NALP12, human NALP13, murine NALP14, human NALP15, human PY10, murine PY16, zebra fish CASPY1, zebra fish CASPY2, zebra fish Pycard. In the last line, an emphasis of the sequence position occurring in a consensus sequence is marked.

FIG. 8 represents a family tree of the identified proteins containing PYD domains, the closeness of the degree of relationship being taken into account in this family tree. The family tree therefore shows the presumed divergence from family tree obtained by genomic duplications caused by evolution.

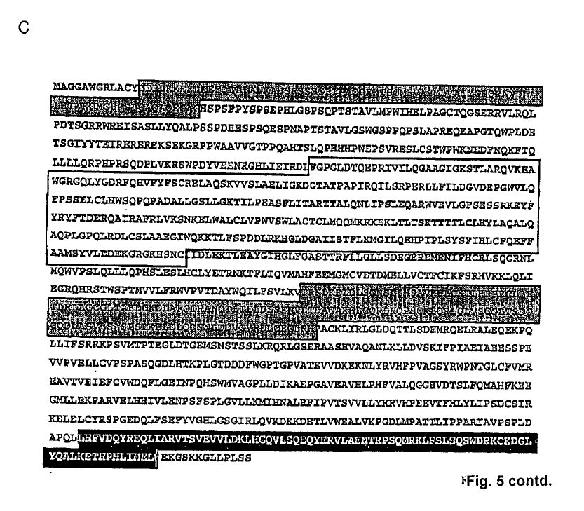

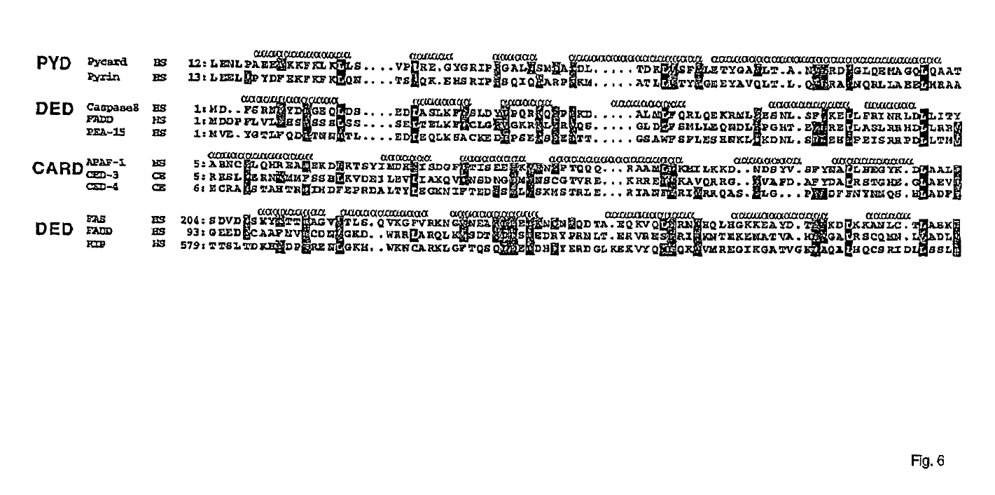

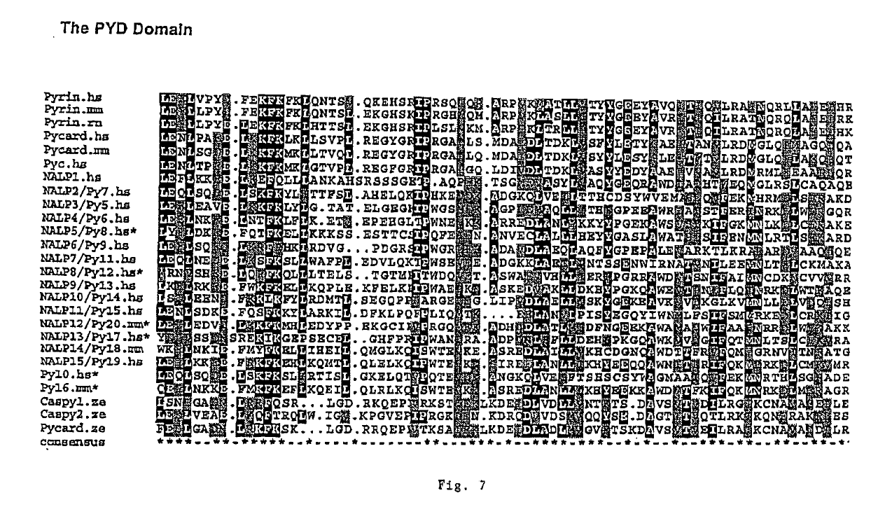

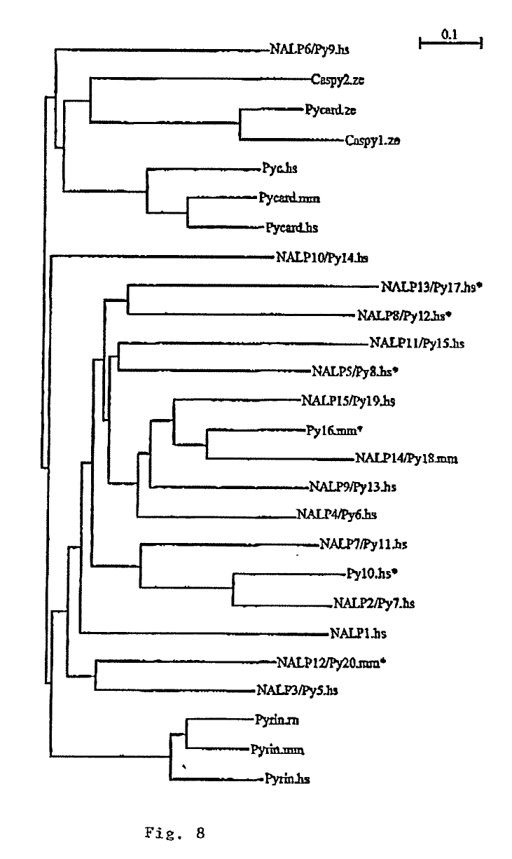

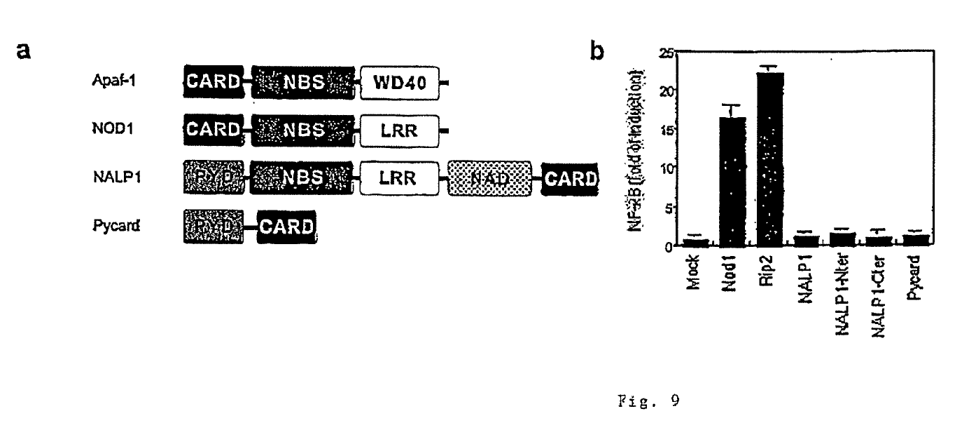

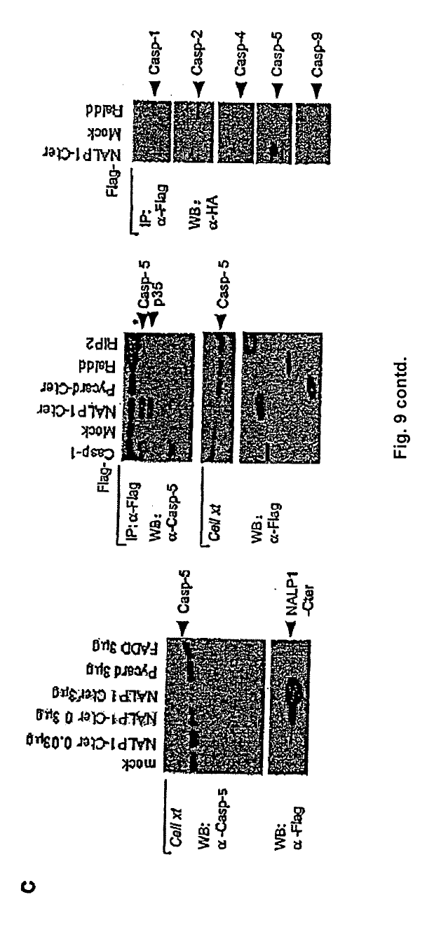

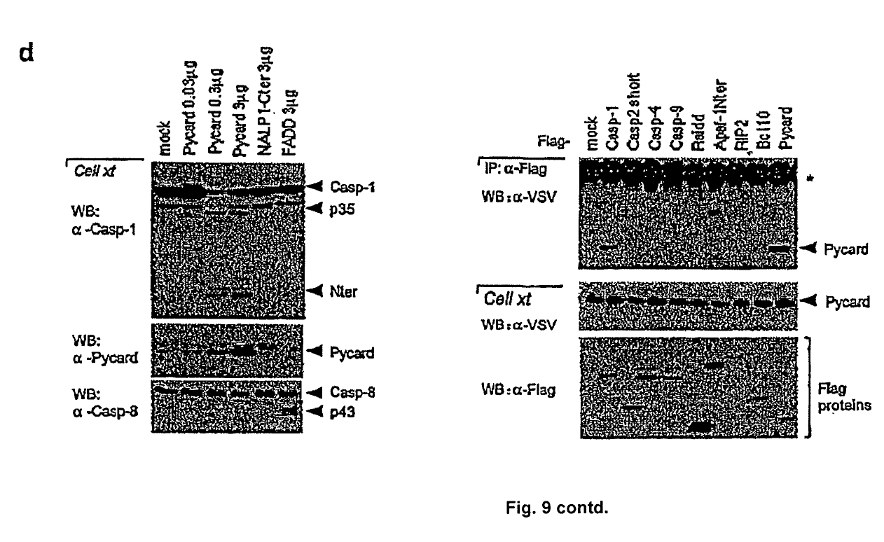

FIG. 9 shows that Pycard and NALP1 are able to associate and activate proinflammotory caspases. FIG. 9a therein represents, in diagrammatic form, the domain structure of Apaf-1, NOD1, NALP1 of the invention and Pycard of the invention (Asc). The two proteins Apaf-1 and NOD1 known from the prior art display a structural relationship with the proteins of the invention. The individual domains with their short names are depicted in FIG. 9a therein, namely the domains CARD, PYD (Pyrin domain), LRR (leucine-rich repeats), NBS (nucleotide binding domain), WD domains and an NAD domain (NALP-associated domain) which occurs in all proteins of the invention of the NALP class and is highly conserved. The NALP1 and Pycard proteins of the invention additionally show in each case the characteristic PYD domain via which the two proteins can interact according to the invention.

FIG. 9b represents the non-involvement of the NALP1 and Pycard proteins of the invention in the activation of NF-κB. For this purpose, 293T cells were transfected with NOD1, RIP2, NALP1, NALP1-Nter, NALP1-Cter and Pycard plasmid constructs, and with a mock vector and with NF-κB luciferase reporter plasmids, and the relative NF-κB transcription activity was measured 24 hours after transfection. It is immediately evident that only the two proteins NOD1 and RIP2 are involved in the induction of NF-κB activation, whereas the proteins of the invention NALP1 (and fragments of NALP1) and Pycard show a transcription activity at the level of the mock vector, i.e. do not activate NF-κB.

FIG. 9c represents in the left-hand plot blots of overexpressed NALP1 Cter (AA 1030 to 1430 which contain the NAD and CARD domain), whereby caspase-5 processing is induced. For this purpose, 293T cells were transfected with 0.5 μg of a caspase-5 plasmid and the indicated amount of NALP1-Cter, Pycard or FADD expression plasmids. In the depicted Western blot, caspase-5 was detected with anti-caspase-5 antibody, and NALP1 with anti-Flag antibody. With an amount of 3 μg of NALP1-Cter expression plasmid, an expression signal of procaspase was scarcely detectable in the cell extract on use of anti-caspase-5 antibodies, i.e. caspase-5 had been very substantially processed. It is evident in the middle plot in FIG. 9c that NALP1 interacts with caspase-5. For this purpose, caspase-5 (2 μg) was coexpressed with 2 μg of the indicated Flag-labeled expression constructs. 24 hours after the transfection, the anti-Flag (M2) immunoprecipitates (IP) were analyzed for the presence of caspase-5, and the cell extracts (xt) were immunoblotted with the indicated antibodies. Whereas no caspase-5 signal was evident after use of anti-caspase-5 antibodies in the cell extract (lower section of the middle figure) on coexpression with NALP1-Cter, the other test mixtures give bands for caspase-5. The corresponding Flag-labeled constructs are detectable by anti-Flag antibodies in all test mixtures (apart from the mock vector). Caspase-5 or p35 is detectable in the immunoprecipitate. p35 is a caspase-5 cleavage product which contains the CARD domain and the p20 caspase subunit. The * in FIG. 9c (middle depiction, top) shows the position of the IgG heavy chain. In the right-hand plot in FIG. 9c, the interaction of the various CARD caspases with NALP1-Cter and the Raidd protein are depicted. The two aforementioned proteins were Flag-labeled and immunoprecipitated with polyclonal anti-Flag antibody. The caspases were detected through their HA (hemagglutinin) label. A band was clearly evident for caspase-5 on addition of NALP1-Cter of the invention, weak signals also for caspase-2 and caspase-4, i.e. the C-terminal CARD domain of NALP1 interacted most strongly with caspase-5. No interaction was observable with caspase-9.

FIG. 9d contains in the left-hand depiction the results of experiments which show that overexpression of Pycard of the invention induces cleavage of caspase-1. For this purpose, the cell extract was investigated in analogy to FIG. 9c (for NALP1-Cter of the invention there). It emerged from this that a marked reduction in the caspase-1 concentration in the cell extract was detectable at elevated Pycard concentrations, as shown in the middle section of

FIG. 9d (left-hand plot) by anti-Pycard antibodies, compared with the other bands, for example on addition of NALP1-Cter or addition of FADD. The right-hand depiction of FIG. 9d shows that Pycard coimmunoprecipitates together with caspase-1. The antibody used is directed against the N-terminal section (CARD) of caspase-1. Overall, the results show that the CARD domain of Pycard strongly interacts with caspase-1 and causes activation thereof. Thus, Pycard of the invention connects NALP1 and caspase-1 as adapter.

FIG. 10 represents the results of experiments which show that combined expression of caspase-1 and caspase-5 induces optimal cleavage of pro-IL1β. In this case, as depicted in FIG. 10a, 293T cells were cotransfected with equal amounts of activated caspase-1 and caspase-5 expression constructs together with either blank vectors or expression constructs of NALP1-Cter and Pycard. The caspase-induced cleavage of pro-ILβ after aspartate 116 was detected using antibodies which are able specifically to bind the p17 cleavage product (IL-1β*) of pro-IL1β. The cell extracts were then immunoblotted in order to be able to determine the expression levels of the transfected proteins. In this case, the Western blot in FIG. 10a shows that with a combination of Pycard, NALPl-Cter, caspase-1 and caspase-5 it is possible to detect IL-1β* with the aid of anti-IL1β* antibodies (see FIG. 10a top). The two sections of the picture in the lower part of FIG. 10a represent the control experiments with anti-Flag and anti-Pycard antibodies. These results correspond to the distribution of the corresponding target proteins in the four test mixtures. The caspase-5 cleavage product p35 is clearly visibly detectable with anti-caspase-5 antibody only in the right-hand lane of the plot in FIG. 10a.

FIG. 10b corresponds to FIG. 10a apart from the facts that equal amounts of the activators NALP1-Cter and Pycard were cotransfected with pro-IL1β and that the caspases were each expressed or coexpressed independently (right-hand lane). The cleavage product IL1β* is detectable only in the Western blot in the right-hand lane, i.e. after coexpression, with the appropriate antibodies.

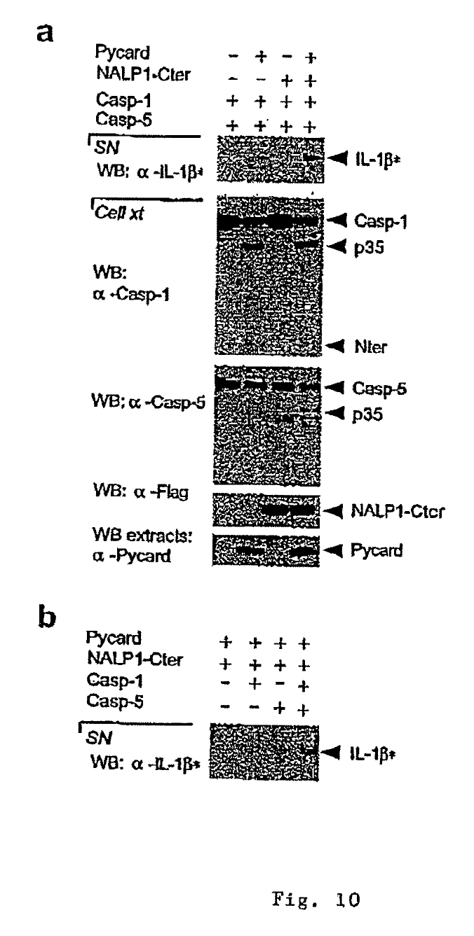

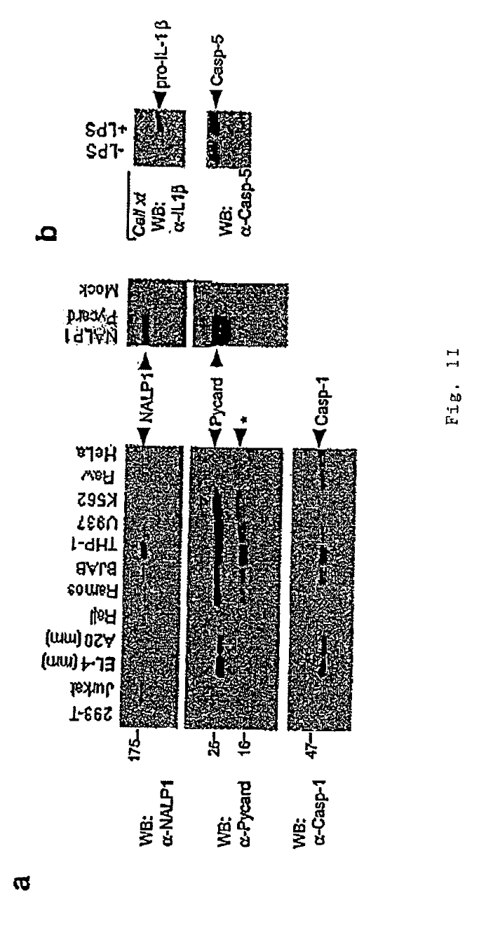

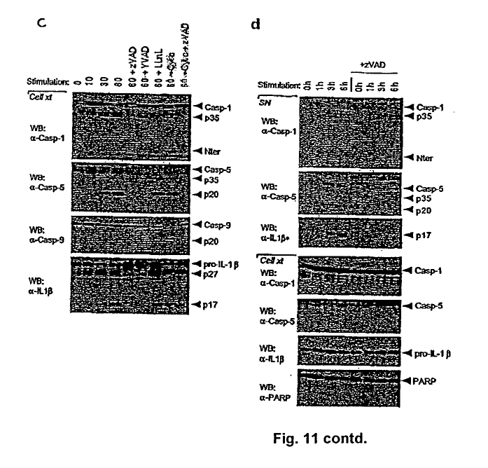

FIG. 11 represents the parallel activation of caspase-1 and caspase-5 during pro-IL1β processing in THP.1 cells, i.e. under physiological conditions. In this case, initially as shown in FIG. 11a the expression of NALP1, Pycard and caspase-1 in various cell lines was investigated by Western blot analysis (293T cells, Jurkat cells, EL4 cells, A20, Raji, Ramos, BJAB, THP-1, U937, K562, Raw, HeLa cells). For this purpose, the cell extracts (30 μg) were mixed with polyclonal antibodies against NALP1 and Pycard and monoclonal antibodies against caspase-1. The afore-mentioned cell lines are all of human origin apart from the lymphocyte cell lines A20 and EL4 which are derived from mice. The * refers to a protein which cross-reacts with the anti-Pycard antibody and which probably represents a shorter, alternative splice version of Pycard. In the right-hand plot of FIG. 11a, a check of the specificity of the antibodies used in this investigation was carried out. For this purpose, 293T cells were transfected with NALP1 and Pycard or a mock expression construct, and the cell lysates were mixed with the appropriate antibody. Overall, the experiments depicted in FIG. 11a showed that THP-1 cells possess strong expression both of NALP1, of Pycard and of caspase-1. They are therefore particularly suitable as objects for investigating the interaction of NALP1, Pycard and caspase-1. The specificity of the antibodies is excellent, as is evident from the right-hand plot in FIG. 11a.

In FIG. 11b, the expression of IL1β and caspase-5 before and after stimulation with LPS has been (1 μg/ml, 1 h, conditions as described below for FIG. 11c, depicted). In this case, after stimulation with LPS, both caspase-5 and pro-IL1β is detectable in the cell extract with the appropriate antibodies.

However, THP1 cells express caspase-5 even without LPS activation. FIG. 11c represents the results of experiments which emerge when cell lysates from THP.1 cells which have been prestimulated with LPS were incubated at 30° C. for the periods shown at the top in FIG. 11c. This is because caspase-1 activation is to be observed spontaneously when cytoplasmic cell extracts are heated to 30° C. The activation of caspase-1, caspase-5, caspase-9 and pro-IL1β, followed by Western blotting in the presence or absence of caspase inhibitors zVADfmk (50 μM), YVADfmk (5 μM) and proteasome inhibitor LLnL (50 μM) was measured as a function of time. Cytochrome C (1 ng) was added to activate Apaf-1 and caspase-9. The monoclonal antibodies employed to detect caspase-5 and caspase-9 each recognize the p20 subunit. The four sections of the picture in FIG. 11c depict the respective bands obtained by labeling with anti-caspase-1 antibody, anti-caspase-5 antibody, anti-caspase-9 antibody and anti-IL1β antibody. It emerged from this that caspase-9 was processed only when cytochrome C was added to the cell extract. Caspase-1 and caspase-5 by contrast show similar kinetics, which is why both are activated by a caspase-9-independent signal transduction pathway. Caspases-1 and -5 are each decomposed into their cleavage products (p20, p35, Nter) time-dependently after activation. Simultaneously with the activation of capsases-1 and -5 (appearance of the cleavage products) it is possible to detect the p17 fragment (active cleavage product of proIL-1β).

Addition of the two caspase-1 inhibitors zVADfmk and YVADfmk blocked proIL-1β activation. Finally, FIG. 11d represents the caspase-1, caspase-5 and pro-IL1β activation after stimulation of the THP1 cells by LPS (10 μg/ml) (instead of increasing the incubation temperature, as in FIG. 11c). The THP1 cells were prestimulated with PMA before addition of LPS. Activation of pro-IL1β, caspase-1 and caspase-5 was measured both in the cell extracts (xt) and in the supernatants (SN). The upper three sections of the picture in this case represent the measurement in the supernatants (SN), and the lower four sections of the picture measurements of the presence in cell extracts. The individual lanes correspond to different stimulation periods (with or without addition of caspase inhibitor ZVAD). The antibodies employed for detection in each case are listed on the left of the sections of the picture, and the corresponding band positions are indicated in particular on the right by appropriate arrows. PARP (lowest section of the picture) is a cleavage product which allows caspase-3 activation to be identified. The cleavage of pro-IL10 was detected by an antibody (anti-IL1β*) which specifically recognizes the cleaved form of pro-IL1β, i.e. IL1β*. Whereas no active forms of caspase-1 or caspase-5 occur in the cell extracts, they are detectable, just like the active form p17 (active cleavage product of proIL-1β), in the supernatants. This means that active forms of caspase-1 and caspase-5 are secreted by the cells together with p17 into the supernatant. A cleavage (activation) of PARP as proapoptotic signal (lowest section of the picture) by contrast does not, as expected, take place.

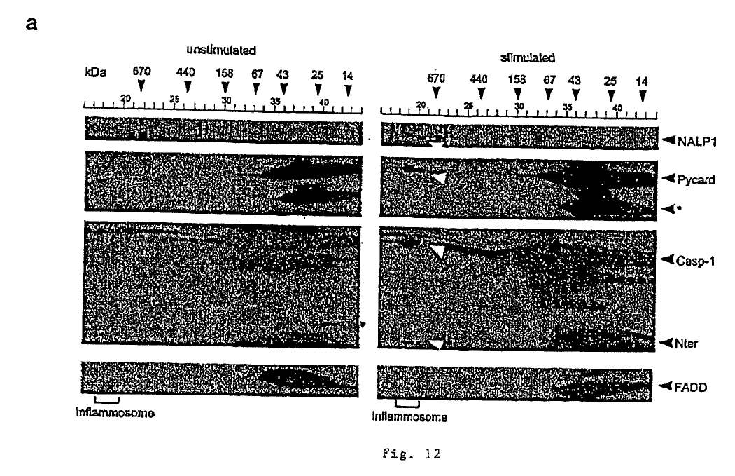

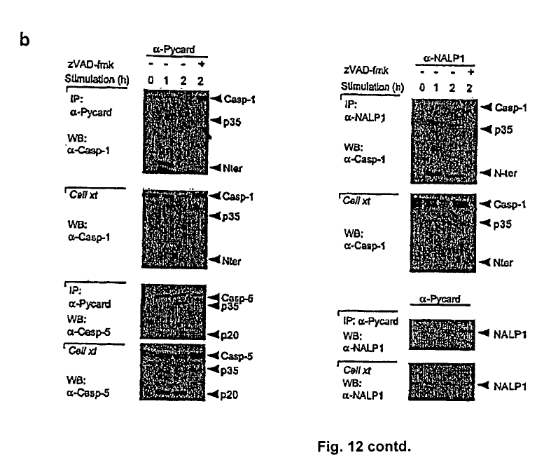

In order to check whether NALP1, caspase-1, caspase-5 and Pycard in fact cooperate to activate proIL-1β, gel filtration methods were used to investigate unactivated cell extracts from THP1 cells. FIG. 12 represents results which show that the formation of a complex comprising NALP1, Pycard, caspase-1 and caspase-5, called the inflammosome, takes place. In this case, the results depicted in FIG. 12a are based on the following experimental mixtures: THP1 cell lysates were incubated at 30° C. for a period of 60 min; these conditions led to spontaneous activation of pro-IL1β (see FIG. 11b). For this purpose, cell extracts were fractionated by size on Superdex S-200 columns. The elution pattern of NALP1, Pycard and capase-1 in the cell extracts before and after activation, i.e. unstimulated and stimulated, is depicted. The white arrows indicate the elution positions of the proteins which are shifted toward a complex of higher molecular weight (imflammosome fractions 19 and 20). The standard is represented in the top line of FIG. 12a (in kDa), i.e. the positioning of correspondingly large proteins in the individual fractions. The various sections of the picture represent the elution profiles for NALP1, Pycard, caspase-1 and FADD (the latter for the purposes of comparison). FIG. 12a shows that NALP1 (with a theoretical molecular weight of 156 kDa) exists as multiprotein complex (about 700 kDa) even without stimulation. After initiation of caspase-1 activation it is possible to observe a distinct shift of the NALP1 complex to a higher molecular weight (about 700 kDa). Moreover Pycard and caspase-1, which in the unstimulated state elute respectively at about 30 kDa and 60 kDa, as expected, are observed after stimulation at least partly also in the 700 kDa fraction containing NALP1. NALP1, Pycard and the two caspases, caspase-1 and caspase-5, form the inflammosome.

FIG. 12b shows that NALP1, Pycard, caspase-1 and caspase-5 form a complex, specifically in a time-dependent manner. In this case, extracts of THP1 cells were stimulated for various periods, as represented in the top line of FIG. 12b, by incubating at 30° C. Immunoprecipitation was effected by anti-Pycard antibodies (left-hand plot) or anti-NALP1 antibodies (right-hand plot). The presence of caspase-1, caspase-5 and NALP1 was then investigated in a Western blot with the aid of the antibodies which are each depicted in FIG. 12b. Once again, the positions of the proteins or protein fragments to be expected there are depicted on the right of the depicted sections of the picture (see above). The result is coimmunoprecipitation of caspase-1 either with Pycard or with NALP1 in THP1 cell extracts in an activation-dependent manner. Essentially the processed (activated) form of caspase-1 was present in the immunoprecipitate, thus differing from the cell extract. Caspase-1 binding is transient because distinctly less caspase-1 was detectable in the inflammosome 2 h after the stimulation. Besides caspase-1, anti-Pycard antibodies also immuno-precipitate caspase-5 and NALP1, depending on the stimulation.

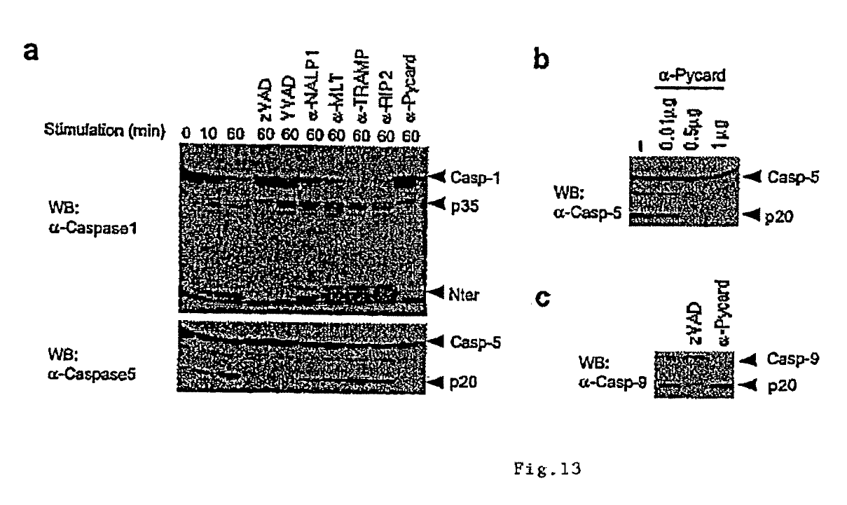

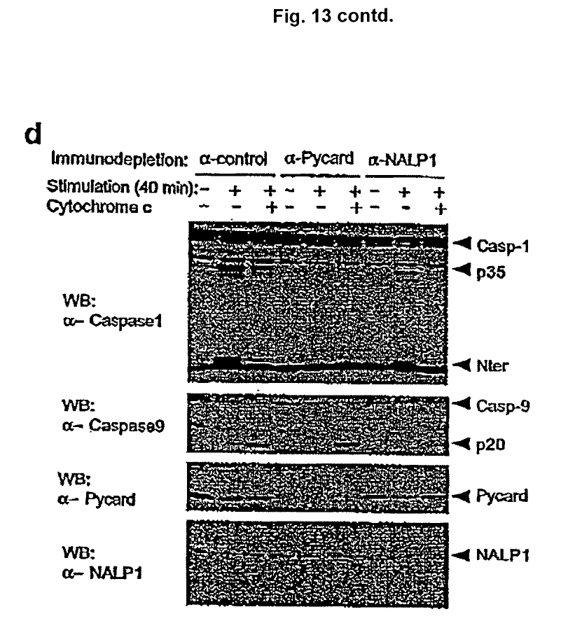

FIG. 13 represents results which show that Pycard (and NALP1) is indispensable for caspase-1 and caspase-5 activation in THP1 cells. For this purpose, as shown in FIG. 13a, THP1 cell lysates were stimulated at 30° C. for various periods and in the presence or absence of antibodies directed for example against Pycard or NALP1 and further control antibodies (MLTparacaspase, TRAMP/DR3, RIP2). The activation of caspase-1 was then followed further as described in FIG. 11b. In the two sections of the picture in FIG. 13a, a Western blot with anti-caspase-1 antibodies is to be seen at the top, and a Western blot with anti-caspase-5 antibodies is to be seen underneath. The results showed that addition of anti-Pycard antibodies to cell extracts immediately downregulates caspase-1 activation, whereas the other antibodies employed as control have no effect. Anti-Pycard or anti-NALP1 antibodies brought about inhibition of caspase-5 activation.

FIG. 13b represents the dose-dependence of the inhibition of caspase-5 activation achieved by anti-Pycard antibodies. These antibodies lead, in appropriate concentrations, to the p20 cleavage product of caspase-5 no longer being detectable in a Western blot with appropriate antibodies. The inhibitory effect of anti-Pycard antibodies was thus dose-dependent.

The results in FIG. 13c confirm that anti-Pycard antibody does not interfere with the cytochrome C-mediated caspase-9 activation (the experimental conditions are described in connection with FIG. 11). This is because the caspase-9 cleavage product p20 is still detectable with anti-caspase-9 antibodies, i.e. no inhibition takes place.

FIG. 13d represents the results of experiments in which THP1 cell extracts were incubated with protein G-adsorbed antibodies directed against Pycard or NALP1 or, as control, Ig. After removal of the corresponding beads, the immunoprecipitate of Pycard and NALP1 was investigated by Western blot analysis. The caspase-1 activation was then brought about by increasing the temperature from 0° to 30° C. In this case, caspase-9 is activated (i.e. p20 is present) only when cytochrome C is added to the samples. If Pycard is removed from the cell extract by precipitation, by adding appropriate antibodies, before the stimulation, the caspase-1 activation was completely blocked. Despite incomplete precipitation of NALP1 with the aid of appropriate antibodies, nevertheless a significant reduction in caspase-1 activation was observed.

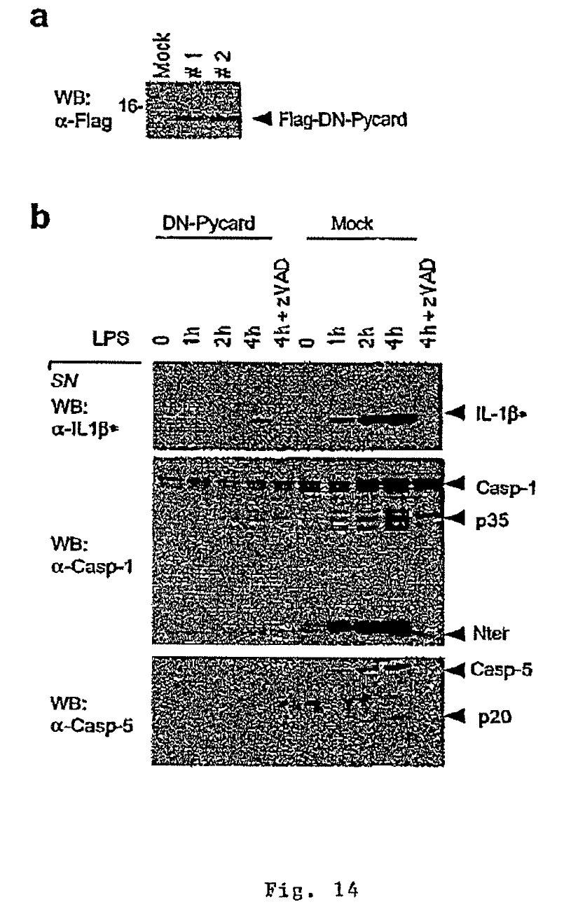

FIG. 14 represents the inhibition of pro-IL1β processing by dominant negative variants of Pycard (DN). As shown in FIG. 14a, THP1 cells were infected by using a retroviral vector which encodes the Flag-labeled Pyrin domain (amino acids 1 to 94, without CARD domain) of Pycard and a puromycin resistance gene. This construct without CARD domain binds to NALP1 but not to caspase-1, and is thus a compound of the invention for blocking NALP1/Pycard induced caspase-1 activation. After selection with puromycin, stably transfected cell populations were investigated for their expression of Flag-labeled proteins by Western blot analysis (two different populations are depicted in FIG. 14a). As shown in FIG. 14a, resulting stably transfected cells which express DN Pycard were treated with LPS (10 μg/ml) for the stated time segments, and the processing of caspase-1, caspase-5 and pro-IL10 was determined in the corresponding cell supernatants (SN), as described in FIG. 11d. DN Pycard was added in the left-hand lanes, and a mock vector as control in the right-hand lanes.

It is clearly evident in the upper section of the picture in FIG. 14b that processed IL1β* appears in the stated stimulation periods only when appropriate mock constructs were employed, whereas only very weak signals were observable in the presence of DN Pycard. Also in relation to caspase-1 and caspase-5 there are only small amounts of the processed forms detectable in the case of addition of DN Pycard, i.e. no activation (secretion) of caspases-1 and -5 takes place. By contrast, expression of DN Pycard had no effect on the LPS-induced NF-kb activation or pro-IL1β synthesis.

The appended annex sheets A1 to A9 forms part of the present disclosure.

The present invention is characterized in detail by the following exemplary embodiments:

Exemplary Embodiments

1st Exemplary Embodiment

In order to check whether the PYD domains—as well as the DD, DED, CARD domains—have the property of interacting only with members within their own family of “6-helix bundle proteins”, expression vectors for PYD proteins were produced and checked for their suitability for interacting with other proteins in coimmunoprecipitation experiments. For this purpose, Pycard constructs were amplified by PCR techniques from the following IMAGE-EST clones: AA528254(965955) and AI148558(1714818). Pycard was amplified using the following primers: JT1509 5′-ATGGGGCGCGCGCGCGAC-3′ and JT1512 5′-TCAGCTCCGCTCCAGG-3′. The PYD domain of Pycard was amplified using JT1509 and JT1510 5′CGACTGAGGAGGGGCC-3′.

NALP1 constructs were amplified with PCR methods using the KIAA0926-EST clones from the Kazusa DNA research institute as template. NALP1 PYD was amplified using JT1497 5′-ATGGCTGGCGGAGCCTGGGGCCGCCTGGCCTGTTACTTG-3′ and JT1525 5′-GATCCGAGGGCATTAGCAC-3′. NALP1 CARD was amplified using

JT1500 5′-GTTGATACTTCAGCTGCTGAGTGGCAGGAG-3′
and
JT1527 5′-GATGAGACTCTGGTGTGG-3′.

The amplified fragments were ligated into PCR-Zero-Blunt (from Invitrogen) and subcloned into the EcoR1 cleavage site of VSV or Flag, which comprise the PCR-3 (from Invitrogen) derived vectors, as described by Thome et al. (1999, J. Biol. Chem., 274, 9962-8). The other constructs employed had already been described previously by Thome et al. (1999 J. Biol. Chem., 274, 9962-8) and, in relation to the description of the experimental procedure, form part of the present disclosure. The publication by Burns et al. (1998, J. Biol. Chem., 273, 12203-12209) describes the procedure for immunoprecipitation. It likewise forms part of the present disclosure and can be described in summary as follows:

293T cells cultivated in DMEM medium which was supplemented with 10% fetal calf serum glutamine were set up with a density of 1-3×10⁶ cells per 10 cm plate and, the next day, transfected with 3 μg of the particular construct by the calcium phosphate precipitation method. The cells were harvested and lysed in lysis buffer 24 to 26 hours after the transfection (the lysis buffer contains 0.2% NP40, 150 mM NaCl, 50 mM EDTA, 30 mM Tris, pH 7.4). The cell lysates were prepurified for at least three hours on Sepharose 6B (from Pharmacia) before the precipitation with an equal amount of proteins at 4° C. for 4 hours with 3 μl of a Flag-agarose (from Kodak International Biotechnology) from 3 μl of Sepharose 6B beads. The resin was washed six times in lysis buffer and, after the last washing, bound protein was eluted by boiling in sample buffer, separated by SDS-PAGE and transferred to nitrocellulose (from Hybond ECL, Pharmacia) in order subsequently to be able to carry out the Western blotting. The anti-VSV and anti-Flag antibodies originated from Sigma. An HRP-conjugated antibody which specifically detected the heavy chains of murine IgG1 (from Southern Biotechnology Associates) was employed.

As shown in FIG. 4 as result of the present exemplary embodiment, it was possible to detect specific binding of Pycard with the PYD domain of Pycard and NALP when coexpression with VSV-labeled Pycard, Flag-labeled constructs which contain either the PYD domain of Pycard or the PYD domain of NALP1 were coexpressed. By contrast, no interaction of the PYD domain of Pycard with other PYD domains or with death domains, CARD domains or death effector domains were found (the latter is not depicted in FIG. 4). A PYD domain therefore interacts specifically with PYD domains via a protein-protein interaction. The result shown in FIG. 4 is thus that Pycard homodimerizes with the aid of its PYD domain and interacts with the PYD domain of NALP1.

2nd Exemplary Embodiment

NALP1 Cter (AA 1030 to 1430, corresponding to the NAD domain and the CARD domain) was amplified with the aid of JT1658 (5′-aaactcctggacgtgagcaag-3′) and JT1500 (5′-tcagctgagtggcaggag-3′) and subcloned in the mammalian expression vector pCR3 in the appropriate reading frame with the tag label. In a similar way, NALP1 Nter (AA 1 to 665, corresponding to the Pyrin domain and the NBS domain) was amplified with the aid of the primers JT1497 (5′-atggctggcggagcctggggc-3′) and JY1526 (5′-caggcctagtattccata-3′). The expression constructs for caspase-4, caspase-1 and caspase-9, Flag-labeled RIP2, Apafl, RAIDD, Bcll0, IL-1β, were provided in accordance with the description in Thome et al. (Current Biology 8, 885 (1998) and Thome et al. (J. Biol. Chem. 274, 9962 to 9968 (1999)). The plasmids which code for caspase-5 and NOD1 originate respectively from Christoph Fröhlich and Gabriel Nunez (Department of Pathology, Univ. of Michigan Med School, 1500 E. Medical Center, Ann Arbor, Mich. 48109, USA).

Transient transfection of 293T cells, cell lysis, immunoprecipitation analysis, immunoblotting and the NF-KB assay were carried out as described by Thome et al. (Current Biology 8, 885 (1998)), to which express reference is made and which is incorporated in the disclosure by reference. The aforementioned methods were carried out as described in the previous citation apart from the use of Ig heavy chain-specific antibodies (HRP-conjugated goat anti-mouse IgG1 and goat anti-rabbit IgG as secondary reagent in the Western blotting (Southern Biotechnology, Birmingham, GB).

Polyclonal antibodies were produced by injecting MAP peptides which correspond to amino acids 2 to 25 of NALP1, to amino acids 2 to 27 of Pycard, into rabbits (Eurogentec, Belgium) and subsequent immunopurification on the corresponding peptides. The monoclonal antibody directed against the CARD domain of caspase-1 originates from Junying Yuan (Boston, Mass. 02115, USA, Harvard Medical School, 240 Longwood Av.).

The other antibodies were purchased from the following manufacturers: caspase-5 (MBL), caspase-9, PARP, cleaved IL-1β D116 (Cell Signaling), anti-Flag antibody (M2, Sigma), anti-VSV antibody (P5D4, Sigma), caspase-3 (Transduction Laboratories).

For in vitro caspase-1/pro-IL10 activation, THP.1 cells were grown in suspension in appropriate bottles in RPMI 1640 medium supplemented with 10% heat-inactivated fetal bovine serum, 50 μM β-mercaptoethanol and penicillin/streptomycin (100 μg/ml each) to a density of 1.5×10⁶ cells/ml and prestimulated with LPS (1 μg/ml) for one hour. Cytosolic extracts were produced as described by Liu et al. (Cell 86, 147-157, 1996). Concerning this, it can be said in summary that the cells were washed in phosphate-buffered saline, were able to swell in 5 volumes of ice-cold hypotonic buffer W (20 mM Hepes-KOH, pH 7.5, 10 mM KCl, 1.5 mM MgCl_{2, 1} mM Na EDTA, 1 mM Na EGTA, and 0.1 mM PMSF) with addition of a protease inhibitor cocktail (Roche, Basle, CH). After cooling in ice for 15 min, the cells were disrupted by passing through a G22 needle 15 times. After centrifugation, the supernatants were filtered (0.45μ) and used for the in vitro IL-1β cleavage assay.

The immunoprecipitation of endogenous caspase-1/NALP1, Pycard interaction complex was carried out with the aid of 5×10⁸ THP.1 cells per time point. The in vitro inflammosome activation was carried out as described above. Immunoprecipitation was carried out with 3 μg of the indicated antibody at 4° C. in buffer W, with 20 μl of protein A Sepharose CL-4B (Pharmacia) for 4 hours. The complexes were recovered by centrifugation and washed 6 times with buffer W. For immunoprecipitation of Pyrin and NALP1, THP.1 cell extracts were incubated with antibodies adsorbed onto protein G beads on ice for 1 hour. After removal of the beads, the caspase-1 activation was caused by increasing the temperature to 30° C.

The activated samples (incubated at 30° C.) or the unactivated samples (left at 4° C., control samples) were loaded onto Superdex-200 HR 10/30 columns, and the proteins were eluted in buffer W at a flow rate of 0.5 ml/min, as 0.5 ml fractions. The Western blotting was carried out after chloroform:methanol precipitation of the complete fraction. The column was calibrated with the following proteins as standard: thyroglobulin (669 kDA), ferritin (440 kDa), catalase (232 kDa), aldolase (158 kDa), bovine serum albumin (67 kDa), ovalbumin (43 kDa), chymotrypsinogen A (25 kDa) and ribonuclease A (13.7 kDa).

In order to achieve LPS activation of the caspase-1/pro-IL-1β activation, THP.1 cells were differentiated with 0.5 μM PMA (Calbiochem) for a period of 3 hours. The cells were washed and plated out on 24-well plates at a density of 4×10⁵ cells per well and left there in order to be able to adhere overnight. After washing in medium without FCS, the cells were treated with LPS 10 μg/ml (E. coli 055:B5, Sigma), as shown in FIG. 11, or not treated. The cell supernatants and cell precipitates were removed and analyzed by Western blotting for various caspases and IL-1β.

In order to produce stable cell lines, Flag-labeled dominant negative (DN) forms of Pycard (AA 1 to 94, corresponding to the Pyrin domain) were cloned into MSCV puromycin-selectable retroviral vectors (Clontech), and a recombinant virus was obtained and titrated after transfection of 293T cells in combination with a vector containing the viral structural genes (VSV-G pseudotyping vector). THP.1 cells were infected, selected with puromycin (5 μg/ml) for a period of 2 weeks, and the cell populations were analyzed for protein expression, caspase-1, caspase-8 and IL-1β activation.

### Annexes A1 to A9

A1:

In proapoptotic signal transduction cascades, the interaction between the various initiator units such as, for example, the death receptor Fas, the various adaptor proteins and the caspases is primarily mediated by three structurally related protein-protein domains, namely the death domain (DD), death effector domain (DED) and the caspase recruitment domain (CARD). It is demonstrated in the present case that a fourth related domain, called the Pyrin domain (PYD), exists. The PYD is observed in Pyrin, a protein which is mutated in patients with familial Mediterranean fever, in Pycard, a regulator of etoposide-mediated apoptosis, in a zebra fish caspase and in two new proteins (NALP1, NALP2) which are structurally related to the apoptosis regulation protein Card4/Nod1. It has been demonstrated for the PYD domain of Pycard that it homodimerizes and interacts with PYD of NALP1. Identification of the PYD family members can in this connection contribute to short-term characterization of proapoptotic and/or proinflammatory signal transduction pathways.

Introduction:

Apoptosis or programmed cell death is an essential process in animals and plants, especially for eliminating unwanted cells in an ordered manner. In recent years, a considerable advance has been achieved in the identification and characterization of the modular nature of molecules responsible for the regulation and execution of apoptosis (Aravind et al., 1999, Hofmann, 1999). Of particular significance in this connection is the fact that three families of homology domains, the death domain (DD), the death effector domain (DED) and the caspase recruitment domain (CARD), which are remotely related to each other, exist and form a superfamily of “six-helix bundle” protein interaction domains. With these domains it is particularly important that they carry out a highly specific interaction with members of the same subfamily and, in most cases, also play a part in the signal transduction process leading to apoptosis and/or inflammation. It should be noted in particular that the “adaptor level” of proteins which specifically transport the death receptor signals to the caspases is heavily occupied by proteins which display combinations of DD/DED/CARD domains. Because of the great importance and the predictability of their function, several systematic searches for new proteins which contain these domains have been carried out successfully. These made it possible to discover proteins such as FLIP, CARDIAK/RIP2, ARC, Bcl10, DEDD (Irmler et al., 1997; Koseki et al., 1998; McCarthy et al., 1998; Stegh et al., 1998; Thome et al., 1999). We report hereinafter on the discovery of a new type of domain, which is called PYD (Pyrin domain) hereinafter. This can be referred to as a fourth subfamily within the “six-helix bundle interaction domains”, specifically because of sequence homologies, structure prediction and interaction properties.

Results and Discussion

Identification of a New Domain: the Pyrin Domain

During analysis of the sequence of the recently identified protein Pycard with a CARD domain (PYD- and CARD-containing protein), which is also known as ASC (apoptosis-associated speckle-like protein), it was realized that the second structural domain which occurs at the N terminus of Pycard (Pyrin domain, PYD) has a weak but significant sequence homology to a large number of other proteins (FIG. 1A). Pycard is a 22 kDA protein which forms aggregates as soon as apoptosis is induced by certain antitumor substances (Masumoto et al., 1999).

A further finding to be made is that in cells forced to express reduced amounts of Pycard there is likewise a significant depression in etoposide-mediated apoptosis. A simple BLAST search using PYD of Pycard revealed that two additional PYD-containing proteins are present in the sequence database, namely Pyrin and Caspy. Pyrin was initially identified as a product of the MEFV (Mediterranean fever) gene which is mutated in patients with familial Mediterranean fever (FrenchFMFConsortium, 1997; InternationalFMFConsortium, 1997), a hereditary periodic fever syndrome which is characterized by episodic fever and serosal and synovial inflammation. The state of knowledge relating to Pyrin is essentially based on its domain structure which, in addition to the PYD domain, also includes a B box zinc finger and a “Spry” domain (FIG. 1B). It has therefore been proposed that Pyrin is a member of the RoRet gene family of nuclear transcription factors, which gave rise to speculation that the protein acts as a transcriptional regulator of inflammation (Centola et al., 1998). However, more recent results suggest that Pyrin is localized in the cytoplasm and has no detectable transcriptional activity (Chen et al., 2000; Tidow et al., 2000). The exact function of Pyrin in inflammatory disorders is therefore still unexplained.

On the basis of the PYD sequence of these two proteins, a general PYD profile was drawn up (Buchner et al., 1996) and employed in the subsequent search steps in the EST database. Two additional PYD-containing proteins NALP1 and NALP2 (NACHT-, LRR- and PYD-containing proteins) were identified. Sequence analysis revealed that the NALPs have a PYD-, NACHT- and LRR-modular organization (FIG. 1A, B). The NACHT and LRR domain architecture is found in proteins involved in inflammation or apoptosis, especially in CARD4/Nod1 (FIG. 1B), an NF-kB-inducing molecule (Bertin et al; 1999; Inohara et al., 1999), a neuronal apoptosis inhibitory protein, NAIP, and the MHC class II transcription activator CIITA (Koonin and Aravind, 2000). It is of interest that the PYD domain of NALP2 is replaced by CARD in CARD4/Nod1, while the overall structural organization is conserved, suggesting a similar functionality (FIG. 1B). CASPY is a PYD- and caspase domain-containing protein which was initially identified by a database search for zebra fish homologues of mammalian apoptosis regulators (Inohara and Nunez, 2000). This caspase has the greatest homology with caspase-13, which in humans contains CARD in place of PYD. It is noteworthy that the same investigation identified a Pycard-related protein in zebra fishes, indicating a high evolutionary conservation of these proteins (FIG. 1A).

The Pyrin Domain is Related to the DD Family

It has been proposed on the basis of sequence comparisons that the DD, DED and CARD domains are structurally related protein-protein interaction modules (Hofmann et al., 1997). All three types of domain have a similar size, and secondary structure analysis revealed a similar arrangement of the six α-helices. All three domains therefore share the property of being able to form homo- or heterodimers and are additionally highly conserved (Hofmann et al., 1997). The structure prediction was later confirmed by NMR analysis of the DDs, DEDs and CARDs (Chou et al., 1998; Eberstadt et al., 1998; Huang et al., 1996; Zhou et al., 1999), because the structural topology of these domains proved to be very similar, especially in relation to the structural core formed by helices α2 to α5 (FIG. 2). Including the PYD-containing sequences in the general alignment against DED, CARD and DD, the PYD domain was identified according to the invention as a potential fourth member of the “DD-folded” superfamily (FIG. 2).

Despite the similarity of their folding, it is known that DD, DED and CARD interact exclusively with members of their subfamily, so that no promiscuity between the domains was detectable. In order to check whether the PYDs have the same properties, expression vectors for the PYD proteins were generated and tested in coimmunoprecipitation experiments for their property of interacting with other proteins. As depicted in FIG. 3, a specific binding of Pycard with the PYDs of Pycard and NALPI was detected when coexpression was carried out with a VSV-labeled Pycard, Flag-labeled constructs containing the PYD of Pycard or the PYD of NALP1. No interactions of the PYD of Pycard with other PYDs or with DDs, CARDs or DEDs (FIG. 3) were detected. The PYD domain is therefore a protein-protein interaction domain which specifically interacts with PYD domains.

Pycard with its PYD CARD two-part domain organization is reminiscent of the DD- and DED-containing molecule FADD or the DD- and CARD-containing protein RAIDD. It is known for both proteins that they adapt a DD-containing protein with a DED or CARD domain-containing protein. For example, the DD of Fas requires FADD in order to bind to the DED of caspase-8. Our results suggest that Pycard represents a new adaptor molecule which couples NALP1 to an as yet unknown and to be defined CARD-containing protein. In fact, initial results show that the CARD of caspase-5 is the target structure of Pycard CARD. The physiological role of this interaction is currently under investigation.

In summary, it must be stated that we have identified PYD as a new protein-protein interaction module which satisfies all the criteria of a member of the DD folding family. Comparable with the limited interaction ability applying to other members, PYDs interact only with PYDs and not with members of the three other subfamilies. In addition, PYDs are found in association with proteins involved in apoptosis and in inflammation, which is demonstrated best by the caspase Caspy. PYDs regularly occur together with CARDs such as, for example, NALP1 and Pycard. Identification of the new PYD-containing proteins therefore probably makes it possible to characterize new proapoptotic or proinflammatory signal transduction pathways, as was also the case after the identification of the DD of Fas some years ago (Itoh and Nagata, 1993). We believe that characterization of NALP1 and the Pycard complex is the first step in this direction, which will finally lead to a better understanding of the molecular causes of inflammatory diseases.

Methods

Sequence and Structure Analysis

The Blast and Profile algorithms were used (Bucher et al., 1996), which are available on the ISREC server (www.isrec.isb-sib.ch). The secondary structure was predicted by the algorithms of Rost and Sander (1993).

Cloning, Expression and Immunoprecipitation

Pycard constructs were amplified by PCR from the following IMAGE EST clones:

AA528254 (965955) and AI148558 (1714818). Pycard was amplified using the following primers: JT1509 5′-ATGGGGCGCGCGCGCGAC-3′ and JT1512 5′-TCAGCTCCGCTCCAGG-3′. The PYD domain of Pycard was amplified using JT1509 and JT1510 5′-CGACTGAGGAGGGGCC-3′.

NALP1 constructs were amplified by PCR using the KIAA0926 EST clone from the Kazusa DNA research institute as template.

NALP-1 PYD was amplified using
JT1497
5′-ATGGCTGGCGGAGCCTGGGGCCGCCTGGCCTGTTACTTG-3′
and
JT1525
5′-GATCCAGGGCATTAGCAC-3′,
NALP1 CARD was amplified using
JT1500
5′-GTTGATACTTCAGCTGCTGAGTGGCAGGAG-3′
and
JT1527
5′-GATGAGACTCTGGTGTGG-3′.

Amplified restriction fragments were ligated into PCR-Zero-Blunt (Invitrogen) and then subcloned into the EcoR1 cleavage site of VSV or Flag-containing PCR-3 (Invitrogen) derived vectors (Thome et al., 1999). Other constructs which were used corresponded to those already described previously (Thome et al., 1999) subcloned.

The immunoprecipitation was carried out as described previously (Burns et al., 1998). Briefly summarized, 293 T cells were cultivated in DMEM medium which was supplemented with 10% fetal calf serum glutamine, set out in a range of 1-3×10⁶ cells per 10 cm plate and transfected with 3 μg of the indicated constructs by the calcium phosphate precipitation the next day. The cells were harvested and lysed in lysis buffer (0.2% NP40, 150 mM NaCl, 50 mM EDTA, 50 mM Tris, pH 7.4) 24-26 hours after the transfection. The cell lysates were prepurified for at least 3 hours on Sepharose 6B (Pharmacia), specifically before precipitation of an equal amount of proteins at 4° C. for four hours with 3 μl of Flag-agarose (Kodak International Biotechnology) and 3 μl of Sepharose 6B beads. The resin was washed 6× in lysis buffer and, after the last washing step, the bound proteins were eluted by boiling in sample buffer, separated by SDS-PAGE and transferred to nitrocellulose (Hybond ECL, Pharmacia) for the subsequent Western blotting. Both anti-VSV and anti-Flag antibodies were purchased from Sigma. An HRP-conjugated antibody which was able specifically to detect the heavy chain of mouse IgG1 (Southern Biotechnology Associates) was employed.

### REFERENCES

Aravind, L., Dixit, V. M. and Koonin, E. V. (1999) The domains of death: evolution of the apoptosis machinery. TIBS, 24, 47-53.

Bertin, J., Nir, W. J., Fischer, C. M., Tayber, O. V., Errada, P. R., Grant, J. R., Keilty, J. J., Gosselin, M. L., Robison, K. E., Wong, G. H., Glucksmann, M. A. and DiStefano, P. S. (1999) Human CARD4 protein is a novel CED-4/Apaf-1 cell death family member that activates NF-kappaB. J.Biol. Chem., 274, 12955-8.

Bucher, P., Karplus, K., Moeri, N. and Hofmann, K. (1996) A flexible search technique based on generalized profiles. Computer Chem., 20, 3-24.

Burns, K., Martinon, F., Esslinger, C., Pahl, H., Schneider, P., Bodmer, J. L., Di Marco, F., French, L. and Tschopp, J. (1998) MyD88, an adapter protein involved in interleukin-1 signaling. J. Biol. Chem., 273, 12203-12209.

Centola, M., Aksentijevitch, I. and Kastner, D. L. (1998) The heriditary periodic fever syndromes: molecular analysis of a new family of inflammatory diseases. Hum. Mol. Genet., 7, 1581-8.

Chen, X., Bykhovskaya, Y., Tidow, N., Hamon, M., Bercovitz, Z., Spirina, O. and Fischel-Ghodsian, N. (2000) The familial mediterranean fever protein interacts and colocalizes with a putative Golgi transporter. Proc. Soc. Exp. Biol. Med., 224, 32-40.

Chou, J. J., Matsuo, H., Duan, H. and Wagner, G. (1998) Solution structure of the RAIDD CARD and model for CARD/CARD interaction in caspase-2 and caspase-9 recruitment. Cell, 94, 171-80.

Eberstadt, M., Huang, B., Chen, Z., Meadows, R. P., Ng, S. C., Zheng, L., Lenardo, M. J. and Fesik, S. W. (1998) NMR structure and mutagenesis of the FADD (Mort1) death-effector domain. Nature, 392, 941-5.

FrenchFMFConsortium. (1997) A candidate gene for familial Mediterranean fever. The French FMF Consortium. Nat. Genet., 17, 25-31.

Hofmann, K. (1999) The modular nature of apoptotic signaling proteins. Cell. Mol. Life. Sci., 55, 1113-28.

Hofmann, K., Bucher, P. and Tschopp, J. (1997) The CARD domain: a new apoptotic signalling motif. Trends Biochem. Sci., 22, 155-156.

Huang, B., Eberstadt, M., Olejniczak, E. T., Meadows, R. P. and Fesik, S. W. (1996) NMR structure and mutagenesis of the Fas (APO-1/CD95) death domain. Nature, 384, 638-41.

Inohara, N., Koseki, T., del Peso, L., Hu, Y., Yee, C., Chen, S., Carrio, R., Merino, J., Liu, D., Ni, J. and Nunez, G. (1999) Nod1, an Apaf-1-like activator of caspase-9 and nuclear factor-kappaB. J. Biol. Chem., 274, 14560-7.

Inohara, N. and Nunez, G. (2000) Genes with homology to mammalian apoptosis regulators identified in zebrafish. Cell Death Differ, 7, 509-10.

InternationalFMFConsortium. (1997) Ancient missense mutations in a new member of the RoRet gene family are likely to cause familial Mediterranean fever. The International FMF Consortium. Cell, 90, 797-807.

Irmler, M., Thorne, M., Hahne, M., Schneider, P., Hofmann, K., Steiner, V., Bodmer, J. L., Schroter, M., Burns, K., Mattmann, C., Rimoldi, D., French, L. E. and Tschopp, J. (1997) Inhibition of death receptor signals by cellular FLIP. Nature, 388, 190-195.

Itoh, N. and Nagata, S. (1993) A novel protein domain required for apoptosis. Mutational analysis of human Fas antigen. J. Biol. Chem., 268, 10932-7.

Koonin, E. V. and Aravind, L. (2000) The NACHT family—a new group of predicted NTPases implicated in apoptosis and MHC transcription activation. TIBS, 25, 223-4.

Koseki, T., Inohara, N., Chen, S. and Nunez, G. (1998) ARC, an inhibitor of apoptosis expressed in skeletal muscle and heart that interacts selectively with caspases. Proc. Natl. Acad. Sci. USA, 95, 5156-60.

Masumoto, J., Taniguchi, S., Ayukawa, K., Sarvotham, H., Kishino, T., Niikawa, N., Hidaka, E., Katsuyama, T., Higuchi, T. and Sagara, J. (1999) ASC, a novel 22-kDa protein, aggregates during apoptosis of human promyelocytic leukemia HL-60 cells. J. Biol. Chem., 274, 33835-8.

McCarthy, J. V., Ni, J. and Dixit, V. M. (1998) RIP2 is a novel NF-kappaB-activating and cell death-inducing kinase. J. Biol. Chem., 273, 16968-16975.

Rost, B. and Sander, C. (1993) Secondary structure prediction of all-helical proteins in two states. Protein Eng., 6, 831-6.

Stegh, A. H., Schickling, O., Ehret, A., Scaffidi, C., Peterhansel, C., Hofmann, T. G., Grummt, I., Krammer, P. H. and Peter, M. E. (1998) DEDD, a novel death effector domain-containing protein, targeted to the nucleolus. EMBO J., 17, 5974-86.

Thome, M., Martinon, F., Hofmann, K., Rubio, V., Steiner, V., Schneider, P., Mattmann, C. and Tschopp, J. (1999) Equine herpesvirus-2 E10 gene product, but not its cellular homologue, activates NF-kappaB transcription factor and c-Jun N-terminal kinase. J. Biol. Chem., 274, 9962-8.

Tidow, N., Chen, X., Muller, C., Kawano, S., Gombart, A.F., Fischel-Ghodsian, N. and Koeffler, H.P. (2000) Hematopoietic-specific expression of MEFV, the gene mutated in familial Mediterranean fever, and subcellular localization of its corresponding protein, pyrin. Blood, 95, 1451-5.

Zhou, P., Chou, J., Olea, R. S., Yuan, J. and Wagner, G. (1999) Solution structure of Apaf-1 CARD and its interaction with caspase-9 CARD: a structural basis for specific adaptor/caspase interaction. Proc. Natl. Acad. Sci. USA, 96, 11265-70.

Figure Legends

FIG. 5(A) Multiple alignment of the Pyrin domain. Position with more than 50% identical or similar amino acids are depicted respectively on black or gray background. The species abbreviation is as follows: HS, Homo sapiens and DR, Danio rerio. Genebank/EMBL access numbers are: AF310103 for human Pycard, AF310104 for murine Pycard, 015553 for Pyrin, AF310105 for NALP1; AF310106 for NALP2, AAF66964 for zebra fish CASPY, AAF66956 for zebra fish Pycard. (B) Domain structure of the proteins which contain a Pyrin domain. Homology domains are named as follows: PYD for Pyrin domain, CARD for caspase recruitment domain; NACHT for NAIP, CIITA, HET-E and TP1 domain; LRR for leucine-rich repeats, SPRY for domain in the SPla and ryanodine receptor. B for B box. (C) Amino acid sequence of NALP1. The various shadings of the boxes correspond to the domains as shown in FIG. 1B (4B).

FIG. 6 Alignment of the representative Pyrin domains (PYD), death effector domains (DED), caspase recruitment domains (CARD) and death domains (DD); this shows the similarity of these interaction domains. α lines indicate the predicted α-helices for PYD (Rost and Sander, 1993) and the indicated α-helices in the DD, CARD and DED solution structures (Eberstadt et al., 1998; Huang et al., 1996; Zhou et al., 1999)

FIG. 4 Pycard homodimerizes with the aid of its PYD and interacts with PYD of NALP1. Flag-labeled constructs contain: PYD of Pycard (Pycard PYD), RAIDD, CARD of Apaf-1 (Apaf1 CARD), PYD of NALP1 (NALP1 PYD), CARD of NALP1 (NALP1 CARD) and a blank vector (mock vector) were cotransfected into 293 T cells with a VSV-labeled Pycard construct. The cells were lysed 24 hours after transfection, and the anti-Flag immunoprecipitates were analyzed in relation to the presence of a VSV-Pycard.

The expression of the various constructs was analyzed in the cell lysates (lower depiction).

1. A DNA sequence, characterized in that it codes for a protein having at least one PYD domain, including all functionally homologous derivatives, fragments or alleles.

2. The DNA sequence as claimed in claim 1, characterized in that a significance level of p<10⁻² results when the PYD domain of the DNA sequence is compared with a search profile as shown in FIG. 3.

3. The DNA sequence as claimed in claim 1 or 2, characterized in that the gene product thereof comprises one of the amino acid sequences (for a PYD domain) as represented in FIG. 6, including all functionally homologous derivatives, alleles or fragments.

4. The DNA sequence as claimed in any of the aforementioned claims, characterized in that it comprises one of the (c)DNA sequences indicated in FIG. 1.

5. The DNA sequence as claimed in any of the aforementioned claims, characterized in that the gene product thereof comprises one of the amino acid sequences indicated in FIG. 1.

6. An expression vector, characterized in that it comprises a DNA sequence as claimed in any of claims 1 to 5.

7. A host cell, characterized in that it is transformed with an expression vector as claimed in claim 6.

8. The host cell as claimed in claim 7, characterized in that it is a mammalian cell, in particular a human cell.

9. A purified gene product, characterized in that it is encoded by a DNA sequence as claimed in any of claims 1 to 5.

10. The purified gene product as claimed in claim 9, characterized in that it is a polypeptide.

11. The purified gene product as claimed in claim 9 or 10, characterized in that it comprises one of the amino acid sequences (for a PYD domain) indicated in FIG. 7, including all functionally homologous alleles, fragments or derivatives.

12. An antibody, characterized in that it recognizes an epitope on a gene product as claimed in any of claims 9 to 11.

13. The antibody as claimed in claim 12, characterized in that it is monoclonal.

14. The antibody as claimed in either of claims 12 or 13, characterized in that it is directed against a sequence section on the PYD domain as epitope.

15. A method for isolating gene products having at least one PYD domain, characterized in that host cells as claimed in claim 7 or 8 are cultivated under suitable conditions promoting expression, and the gene product is finally purified from the culture.

16. A method for expressing gene products having at least one PYD domain, characterized in that host cells are transformed with an expression vector as claimed in claim 6.

17. The use of a DNA sequence as claimed in any of claims 1 to 5 or of a gene product as claimed in any of claims 9 to 11 for the treatment of disorders based on dysregulation of intracellular signal transduction.

18. The use of a DNA sequence or of a gene product as claimed in claim 17, characterized in that the disorder is one with an excessive inflammatory response.

19. The use of a DNA sequence or of a gene product as claimed in claim 17 or 18, characterized in that the disorder is psoriasis, artheriosclerosis, bacterial or viral infectious diseases, in particular bacterial or viral meningitis or bacterial pneumonia, multiple sclerosis, rheumatoid arthritis, asthma, sarcoidosis, glomerular nephritis or osteoarthritis.

20. A compound, characterized in that it blocks the specific interaction of PYD domains for intracellular signal transmission.

21. The compound as claimed in claim 20, characterized in that it is an organic chemical compound having a molecular weight of, preferably, <3000.

22. The compound as claimed in claim 21, characterized in that it passes through the cell membrane by diffusion or via membrane transport proteins.

23. The use of a compound as claimed in claim 20 or 21 for the treatment of (and for the production of a drug product for the treatment of) psoriasis, artheriosclerosis, bacterial or viral infectious diseases, especially bacterial or viral meningitis or bacterial pneumonia, multiple sclerosis, rheumatoid arthritis, asthma, sarcoidosis, glomerular nephritis or osteoarthritis.
